(19) United States
(12) Patent Application Publication
(10) Pub. No.: US 2023/0158495 A1
(43) Pub. Date: May 25, 2023

(54) RATIONAL DESIGN OF MICROFLUIDIC PUMPS INCORPORATING ACTIVELY BEATING CILIA

(71) Applicant: University of Southern California, Los Angeles, CA (US)

(72) Inventors: Eva KANSO, Los Angeles, CA (US); Janna NAWROTH, Munich (DE); Feng LING, Los Angeles, CA (US); Kakani Katija YOUNG, Carmel Valley, CA (US); Margaret Jean McFALL-NGAI, Kailua, HI (US); Michael SHELLEY, Los Angeles, CA (US)

(21) Appl. No.: 17/947,837

(22) Filed: Sep. 19, 2022

Related U.S. Application Data
(60) Provisional application No. 63/281,513, filed on Nov. 19, 2021.

(51) Int. Cl. B01L 3/00; G06F 30/28

(52) U.S. Cl. CPC B01L 3/50273; B01L 3/502707; G06F 30/28; B01L 2300/0645; B01L 2400/0493; B01L 2400/043; B01L 2300/16

(57) ABSTRACT

A method for designing a microfluidic device includes steps of: a) receiving an input design of a bare microfluidic channel to which one or more cilia layers are to be added, the bare microfluidic channel having a predetermined cross section, the bare microfluidic channel defining an inner surface and an outer surface, a first direction being a fluid flow direction along a length of the bare microfluidic channel and a second direction perpendicular to the first direction; b) receiving operation parameters for a ciliated microfluidic channel formed from the bare microfluidic channel, the operation parameters including fluid viscosity and an opposing pressure gradient in an adverse direction to the fluid flow direction; and c) determining cilia design parameters for the one or more cilia layers to be attached to and distributed over the inner surface, the cilia design parameters being determined from the incompressible Brinkman equation.

### CROSS-REFERENCE TO RELATED APPLICATIONS

This application claims the benefit of U.S. provisional application Ser. No. 63/281,513 filed Nov. 19, 2021, the disclosure of which is hereby incorporated in its entirety by reference herein.

### STATEMENT REGARDING FEDERALLY SPONSORED RESEARCH OR DEVELOPMENT

The invention was made with Government support under Contract No. HL153622-01A1 awarded by the National Institutes of Health and under Contract No. 1608744 awarded by the National Science Foundation. The Government has certain rights to the invention.

### TECHNICAL FIELD

In at least one aspect, the present invention relates to microfluidic devices having channels with cilia layers.

### BACKGROUND

Cilia are micro-scale hair-like structures that cover many eukaryotic cells, from single-celled protozoa to mammalian epithelial surfaces. Motile cilia function in both fluid transport across the cell surface as well as in the sensing of environmental cues. The distribution of cilia in animal tissues is highly correlated with their function. In aquatic species, cilia commonly occur along external and internal epithelial surfaces, where they have a broad array of functions, from food capture to acquisition of microbial partners [1,2]. When animals invaded terrestrial habitats, cilia became restricted to internal epithelial surfaces to reduce water loss across mucociliary membranes, rendering them difficult subjects for direct study. In mammals, they serve a number of functions including mucus clearance in the respiratory system, transport of cerebrospinal fluid in the brain, and transport of egg cells in fallopian tubes [3-5].

Currently, microfluidic devices having channels that include artificial cilia are known. Some design utilize artificial cilia composed of a magnetic material which move in response to the movement of an external magnetic field. Although these systems work well, design optimization for a given set of conditions is required.

Accordingly, there is a need for methodology for optimized microfluidic devices having cilia therein, whether artificial or biological cilia.

### SUMMARY

In at least one aspect, a method for designing a microfluidic device performed by a computing device is provided. The method includes a step of receiving an input design of a bare microfluidic channel to which one or more cilia layers are to be added. The bare microfluidic channel has (i.e., defines) a predetermined cross-section. The bare microfluidic channel also defines an inner surface and an outer surface. A first direction is a fluid flow direction along a length of the bare microfluidic channel and a second direction is perpendicular to the first direction. The method also includes a step of receiving operation parameters for a ciliated microfluidic channel formed from the bare microfluidic channel. The operation parameters include fluid viscosity and an opposing pressure gradient in an adverse direction to the fluid flow direction. Finally, the method also includes a step of determining cilia design parameters for the one or more cilia layers to be attached to and distributed over the inner surface. Characteristically, the cilia design parameters can be optimized based on the incompressible Brinkman fluid equation.

In another aspect, a computational model aimed at understanding the capabilities and limitations of flow transport by cilia in a rectangular channel is provided.

The foregoing summary is illustrative only and is not intended to be in any way limiting. In addition to the illustrative aspects, embodiments, and features described above, further aspects, embodiments, and features will become apparent by reference to the drawings and the following detailed description.

### BRIEF DESCRIPTION OF THE DRAWINGS

The patent or application file contains at least one drawing executed in color. Copies of this patent or patent application publication with color drawing(s) will be provided by the Office upon request and payment of the necessary fee.”

For a further understanding of the nature, objects, and advantages of the present disclosure, reference should be made to the following detailed description, read in conjunction with the following drawings, wherein like reference numerals denote like elements and wherein:

FIG. 1A. Schematic flowchart depicting a method for designing a microfluidic device having cilia layers performed by a computing device.

FIGS. 1B-1, 1B-2, 1B-3, and 1B-4. (B-1) cilia that beat synchronously with no metachronal coordination, (B-2) cilia that have weak coordination, (B-3) cilia whose organization has gaps and with overlap in cilia motion, (B-4) cilia whose beating patterns are taken from biological data such as those base on rabbit tracheal cilia. In each subfigure, the characteristic cross-sectional flow is shown to the right.

FIG. 1C. Schematic of a microfluidic channel with a cross sectional area that varies along the flow direction.

FIG. 1D. Schematic of a microfluidic channel with a dynamically variable cross sectional area.

FIG. 1E. Schematic of a computing device/system that can be used to practice the computer implemented method for designing a microfluidic device having cilia layers.

FIGS. 2A, 2B, and 2C. Porous Cilia Model in a 2-D Channel. (A) Discrete Lagrangian particle representation of the quasi-1D longitudinal density wave inspired by cilia motion. Each column of particles are labeled as x_{c}(n,t), and ρ_{c} relates the particle formulation to the continuum model. As time progresses, the traveling wave moves to the left, but generates net fluid motion to the right. (B-C). Cilia velocity ν_{c}(x,t) and Brinkman coefficient ζ_{c}(x,t) as interpolated on the Eulerian grid, respectively. Every row of particles inside the porous layer follow the shown velocity and density profile. Here the increase in darkness indicates the passage of time. ϵ=0.1, and ρ_{c}=H=L=μ=ω=1.

FIGS. 3A, 3B, 3C, 3D, 3E, and 3F. Porous media pump generalizes the envelope model. (A-B). Instantaneous velocity field u(x,y,t) and flow profile Uₓ(y) produced by the envelope model of ciliated duct, where fluid is pumped by slip velocities at the impermeable boundaries. Pumping is maximized without adverse pressure, but even with a relatively small adverse pressure gradient of ΔP/μωL⁻¹=−1, net flow reverses direction. (C-D). When pumping with a porous layer of cilia (h/H=0.2), adverse pressure gradient can only reverse net flow direction in regions where cilia is absent. Density distribution of cilia ρ_{c} divided by the reference density ρ̅_{c} is shown in gray scale. (E-F). the porous media model can model ciliated ducts with long cilia (h/H=0.9) that can also pump fluid against large adverse pressure gradient ΔP. ρ_{c}h=1000 in c-f ΔP/μωL⁻¹=−50 in d,f, and ϵ0.159≈L/2π for all panels.

FIGS. 4A-1, 4A-2, 4B-1, and 4B-2 Cilia density, ciliated fraction, and the material constraint. (A-1, A-2). Net flow speed U as a function of ciliated fraction h/H for increasing values of ρ̅_{c} at zero (left) and finite adverse pressure of ΔP/μωL⁻¹=−50 (right) in grayscale. Dashed line shows the theoretical flow speed limit of [symbol] v_{c} [symbol] ₓ. (B-1, B-2). U(h/H) with different values of constant ρ̅_{c}h in dark blue gradient. Under large adverse pressure, positive flow is only possible at high ciliated fraction for large ρ_{c} and ρ_{c}h. Square marks show the corresponding flow speed for FIGS. 1c-f. ϵ=0.159 and H/L=1 for all panels. Note the scale changes in y-axis (bi-directional log mapping is used where convenient [8]).

FIGS. 5A, 5B, and 5C. Mean flow speed as a function of adverse pressure and lumen aspect ratio. (A). Flow speed U versus ΔP for various h/H; U is linear in ΔP but nonlinear in h/H. (B). When adverse pressure is present, the flow direction at lower ciliated fractions h/H reverses, and the magnitude of the reverse flow is larger as the lumen diameter H increases. (C). For sufficiently tall channel (H>>L), negative ΔP produces a backward flow speed that is quadratic with respect to lumen diameter H, in line with Poiseuille flow in a rectangular channel. ρ̅_{c}h=1000 and ϵ=0.159 for all panels.

FIG. 6. Designing an optimal ciliary pump. For any fixed geometrical design (ciliated fraction h/H and lumen aspect ratio H/L), flow rate and adverse pressure has a trade-off akin to all pumps in general. Thus the most efficient operating point for each geometric design is at the corner of each curve. ρ̅_{c}h=1000 and Ε=0.159.

FIGS. 7A and 7B. Validation of the Brinkman solver. (A). L₂ error convergence of vorticity (black) and velocity (red and blue) for the slip boundary problem, where uₓ(x, 0)=uₓ(x, H)=cos(2πx). Tested on unit square domain using grid size of 20×20 to 200×200 with μ=1, L=H=1, ζ=1000. (B). Convergence plot for Poiseuille flow with μ=1, L=H=1, ζ=16, ΔP=−1. The error in u_{y} is noisy due to floating point precision (no vertical flow).

FIGS. 8A, 8B, 8C, 8D, 8E, 8F, and 8G. Comparison of CDs with ciliary flame versus ciliary carpet design in the model system Bathochordaeus sp. (A). The stomach (St), pharynx (P), and esophagus (E) are lined with ciliary carpets. A ciliated funnel (CF) containing a ciliary flame connects the mouth (M) cavity to the internal blood sinus (S). (B). Confocal image of the ciliary carpet of the esophagus (magenta: cilia, acetylated α-tubulin). Inset: Horizontal cross section. Note that the cilia only fill a small fraction of the lumen. (C). Markers indicating direction and magnitude of fluid flow driven by the esophageal ciliary carpet. (D). Kymograph of the esophageal carpet indicating a beat frequency of ˜20 Hz. (E). Confocal image of the ciliary flame inside the ciliated funnel (turquoise: cilia, acetylated α-tubulin). Inset: Horizontal cross section. Note that the cilia fill a large fraction of the lumen. (F). Markers showing direction and speed of fluid flow converging into the ciliated funnel driven by the ciliary flame. (G). Kymograph of the ciliary flame indicating a beat frequency of ˜60 Hz.

FIGS. 9A and 9B. The morphospace of CDs in nature. (A). CDs plotted based on ciliated fraction h/H and lumen diameter H and classified by their functions and phyla (also see FIG. 4). Here, triangles represent systems whose primary functions are thought to be (ultra-) filtration, excretion, or pressure generation. Circles denote systems that transport and mix food particles, liquids, or cells. The squares represent systems whose fluid transport functions are currently unknown or disputed. The color code indicates the phylogenetic clades listed in the color bar. The dotted line is a linear fit indicating robust correlation between H and h/H in CDs (R-square goodness of fit=0.68). (B). Phylogenetic tree showing the phyla known to feature CDs and included in the analysis (bold type) and the phyla where CDs are absent, not documented, or shaped in a way that cannot be captured by the morphospace in (A). The color code is the same as in (A). Tree design adapted from [54, 55];

FIGS. 10A-1, 10A-2, 10A-3, 10A-4, 10A-5, 10A-6, 10A-7, 10A-8, 10B, 10C, 10D, and 10E. Analysis of CDs with ciliary carpet versus ciliary flame design in Brinkman-Stokes fluid model. (A-1, A-2, A-3, A-4, A-5, A-6, A-7, A-8). Cilia are modeled collectively as an active porous medium of combined ciliated fraction h/H inside an CD with lumen diameter H subject to an adverse pressure gradient ΔP. Prescribed unidirectional traveling density waves ρ_{c}(x, t) in the porous ciliary layers produce instantaneous flows u with cross-sectional flow profile Uₓ and net flow speed U. A direct comparison of u and Uₓ in ciliary carpet (h/H=0.2 labeled I and II) and flame (h/H=0.9 labeled II and IV) designs shows that the carpet design produces more flow at ΔP=0, but under adverse pressure (red arrows), only the flame design pumps fluid in the positive direction. (B). Flow speed U as a function of ciliated fraction h/H under zero and non-zero adverse pressure gradient. The no-slip boundaries imply that both h=0 and h=H yields zero net flow. Under large adverse pressure, positive flow is only possible at high ciliated fraction. (C). Flow speed U versus ΔP for various h/H; U is linear in ΔP but nonlinear in h/H. At larger h/H, U decays more slowly as ΔP increases, hence the ability to produce positive flow at higher adverse pressure. (D). Net flow speed U over the morphospace (H, h/H) at ΔP=0. (E). Adverse pressure gradients ΔP for which a net flow speed of U=100 μm s⁻¹ is achievable. Contour lines and values reveal that flame-like designs are more effective in pumping against a large adverse pressure gradient ρ̅_{c}h=1000 for panel d-e, ω=20 Hz and L=100 μm for a-e, H=100 μm for panel a-c.

FIGS. 11A, 11B, and 11C. Optimal CD designs. (A, B). We maximize the pumping efficiency η∝U³H/ρ̅_{c}h over CD geometries (H, h/H) and adverse pressure gradients ΔP for each ciliary material constant ρ̅_{c}h ∈{1, 5, 10, 100, 1000}. Optimal geometries are shown in solid white lines labeled by the corresponding ρ̅_{c}h value; background orange color represents optimal ΔP* while green color represents the corresponding volume flow rate Q* (* refers to optimal values). Data points shown in FIG. 3.1a are superimposed (symbols). (C). Dotted curves are (Q*,ΔP*) for various material constants ρ̅_{c}h. Black solid curves are pump characteristic curves (see text) of two extreme versions of CDs. Characteristic curves of the larvacean ciliated funnel (CF, blue) and esophagus carpet (EC, pink) (FIG. 2) computed in our model using typical morphological parameters (CF: h/H=0.9, H=50 μm, ρ̅_{c}h=200; EC: h/H=0.05, H=300 μm, ρ̅_{c}h=40, see Methods section below).

FIGS. 12A and 12B. Two different methods were used for measuring duct lumen (DL) diameter and ciliated fraction. (A). In carpet-style ciliated ducts, such as the ciliated esophagus in the giant larvacean Bathochordaeus stygius (own data, c.f FIG. 8A), where cilia are oriented perpendicular to the duct's walls, h/H was determined as the ratio of the ciliary layer height h and the duct lumen diameter H. Since ciliary carpets are assumed to line both “floor” and “ceiling” of the ciliated duct, ciliary length corresponds to 1/2 h. (B). In flame-style ciliated ducts, such as the ciliated funnel of Oikopleura dioica, the cilia are aligned longitudinally to the duct and hence cilia density rather than length determines the ciliated fraction. h/H was hence determined as the ratio of the cross-sectional area of the cilia to the cross-sectional area of the duct lumen. The TEM image was reproduced from FIG. 9 in Holmberg K, Acta Zoologica 63.2 (1982): 101-109 [50], with permission from John Wiley and Sons.

FIGS. 13A, 13B, 13C, 13D-1, 13D-2, 13E-1, and 13E-2. Ciliated ducts in Urochordates and Mollusks feature both carpet and flame designs. (A). The ciliated pharnyx in the giant larvacean Bathochordaeus stygius (Urochordates) is characterized by a ciliary carpet and a low h/H ratio. (B). The ciliated funnel of larvacean Fritillaria sp. (Urochordates) is a flame design. (C). The ciliated conduit of the light organ in the Hawaian Bobtail Squid Euprymna scolopes (Mollusks) features a carpet design in the duct and antechamber regions and a flame-like design in the bottleneck region, as seen in the close-up immunofluorescent and transmission electrode images of (D). the ciliated duct and (E-1, E-2) the bottleneck region.

FIG. 14. Indexed plot of ciliated fraction as a function of lumen diameter. The plotted numbers correspond to the index numbers in Table 1 and hence indicate the animal species corresponding to each data point, as well as the associated source of the data.

FIGS. 15A, 15B, 15C, and 15D. Fits of measured geometric parameters. We show the fitted trend of ciliated fraction h/H vs lumen diameter H in log-linear scale, cilia length h and lumen diameter H in log-log scale, and total duct length vs lumen diameter H in log-log scale. On the other hand, h/H and duct aspect ratio of diameter H over total duct length shows no clear correlation. Here the symbols follow that of FIG. 3.1: º for CDs with confirmed transport/mixing function, ∇ for filtration, and □ for unknown function.

FIGS. 16A, 16B, 16C, and 16D. Porous Cilia Model in a 2-D Channel. From A to D, discrete Lagrangian particle representation of the quasi-1D density wave due to cilia motion in the reference and current configurations. Then we show the isotropically coarse-grained Brinkman drag coefficient ζ(x,y,t)=3πμρ̅_{c}ρ_{c}(x,t)y_{c}(y) and the applied cilia velocity field v(x,y,t)=ν_{c}(x,t)y_{c}(y) in the current configuration on an Eulerian discrete grid. Here ρ̅_{c}=1/b relates the continuum model to the particle formulation. The amplitude and wave number of the density wave are ∈=0.1 and k=1, respectively. As time progresses, the traveling wave moves to the left, but generates net fluid motion to the right (fluid motion not shown).

FIG. 17. Estimated material constraint based on collected data. Histogram of estimated ρ̅_{c} h using data collected in the Methods section. Here we use (h/H)² the measured cilia to lumen area ratio to estimate reference cilia density whenever appropriate, and assumes a baseline cilia density of 50% for carpet-like designs if no images are available.

FIGS. 18A, 18B, 18C, 18D, 18D, 18E, and 18F. (Top row) Mean flow speed as a function of ciliated fraction. in the absence of adverse pressure gradients (ΔP=0), flow speed U decays as a function of ciliated fraction h/H, with the exception that the flow is 0 at h=0 and h=H. When adverse pressure is present, the flow direction at lower ciliated fractions h/H reverses, and the magnitude of the reverse flow is larger as the lumen diameter H increases. Note the change in the scale of the y-axis. (Bottom row) Mean flow speed as a function of lumen diameter. The flow speed U decays until the cilia can no longer maintain forward flow. In the limit of large lumen diameter, any finite negative ΔP causes a backward flow that is quadratic with respect to lumen diameter H, in line with pressure-driven Couette flow in a rectangular channel. Note the scale changes in y-axis (bottom row uses the bi-directional log mapping [101]).

FIGS. 19A-1, 19A-2, 19A-3, 19B-1, 19B-2, and 19B-3. Optimal ciliated duct morphologies at different adverse pressure gradients ΔP. The landscape of power efficiency η(H, h/H) at representative values of ΔP. The optimal values as we vary ΔP (darkest point per plot) connect to form the optimal lines shown in FIG. 8a,b. Here, the material constraint is set to ρ̅_{c}h=1000.

FIGS. 20A-1, 20A-2, 20A-3, 20B-1, 20B-2, and 20B-3. Optimal. Optimal solutions for different values of material constraints ρ̅_{c}h. Here brightness represents degree of optimality, while saturation to red represents the maximal adverse pressure gradient the optimal system can withstand.

FIGS. 21A, 21B, 21C, 21D, 21E, 21F, 21G, 21H, and 21I. Table 1 of cilia parameters.

### DETAILED DESCRIPTION

Reference will now be made in detail to presently preferred embodiments and methods of the present invention, which constitute the best modes of practicing the invention presently known to the inventors. The Figures are not necessarily to scale. However, it is to be understood that the disclosed embodiments are merely exemplary of the invention that may be embodied in various and alternative forms. Therefore, specific details disclosed herein are not to be interpreted as limiting, but merely as a representative basis for any aspect of the invention and/or as a representative basis for teaching one skilled in the art to variously employ the present invention.

It is also to be understood that this invention is not limited to the specific embodiments and methods described below, as specific components and/or conditions may, of course, vary. Furthermore, the terminology used herein is used only for the purpose of describing particular embodiments of the present invention and is not intended to be limiting in any way.

It must also be noted that, as used in the specification and the appended claims, the singular form “a,” “an,” and “the” comprise plural referents unless the context clearly indicates otherwise. For example, reference to a component in the singular is intended to comprise a plurality of components.

The term “comprising” is synonymous with “including,” “having,” “containing,” or “characterized by.” These terms are inclusive and open-ended and do not exclude additional, unrecited elements or method steps.

The phrase “consisting of” excludes any element, step, or ingredient not specified in the claim. When this phrase appears in a clause of the body of a claim, rather than immediately following the preamble, it limits only the element set forth in that clause; other elements are not excluded from the claim as a whole.

The phrase “consisting essentially of” limits the scope of a claim to the specified materials or steps, plus those that do not materially affect the basic and novel characteristic(s) of the claimed subject matter.

With respect to the terms “comprising,” “consisting of,” and “consisting essentially of;” where one of these three terms is used herein, the presently disclosed and claimed subject matter can include the use of either of the other two terms.

It should also be appreciated that integer ranges explicitly include all intervening integers. For example, the integer range 1-10 explicitly includes 1, 2, 3, 4, 5, 6, 7, 8, 9, and 10. Similarly, the range 1 to 100 includes 1, 2, 3, 4 . . . 97, 98, 99, 100. Similarly, when any range is called for, intervening numbers that are increments of the difference between the upper limit and the lower limit divided by 10 can be taken as alternative upper or lower limits. For example, if the range is 1.1. to 2.1 the following numbers 1.2, 1.3, 1.4, 1.5, 1.6, 1.7, 1.8, 1.9, and 2.0 can be selected as lower or upper limits.

When referring to a numerical quantity, in a refinement, the term “less than” includes a lower non-included limit that is 5 percent of the number indicated after “less than.” A lower non-includes limit means that the numerical quantity being described is greater than the value indicated as a lower non-included limited. For example, “less than 20” includes a lower non-included limit of 1 in a refinement. Therefore, this refinement of “less than 20” includes a range between 1 and 20. In another refinement, the term “less than” includes a lower non-included limit that is, in increasing order of preference, 20 percent, 10 percent, 5 percent, 1 percent, or 0 percent of the number indicated after “less than.”

In the examples set forth herein, concentrations, temperature, and reaction conditions (e.g., pressure, pH, flow rates, etc.) can be practiced with plus or minus 50 percent of the values indicated rounded to or truncated to two significant figures of the value provided in the examples. In a refinement, concentrations, temperature, and reaction conditions (e.g., pressure, pH, flow rates, etc.) can be practiced with plus or minus 30 percent of the values indicated rounded to or truncated to two significant figures of the value provided in the examples. In another refinement, concentrations, temperature, and reaction conditions (e.g., pressure, pH, flow rates, etc.) can be practiced with plus or minus 10 percent of the values indicated rounded to or truncated to two significant figures of the value provided in the examples.

For any device described herein, linear dimensions and angles can be constructed with plus or minus 50 percent of the values indicated rounded to or truncated to two significant figures of the value provided in the examples. In a refinement, linear dimensions and angles can be constructed with plus or minus 30 percent of the values indicated rounded to or truncated to two significant figures of the value provided in the examples. In another refinement, linear dimensions and angles can be constructed with plus or minus 10 percent of the values indicated rounded to or truncated to two significant figures of the value provided in the examples.

With respect to electrical devices, the term “connected to” means that the electrical components referred to as connected to are in electrical communication. In a refinement, “connected to” means that the electrical components referred to as connected to are directly wired to each other. In another refinement, “connected to” means that the electrical components communicate wirelessly or by a combination of wired and wirelessly connected components. In another refinement, “connected to” means that one or more additional electrical components are interposed between the electrical components referred to as connected to with an electrical signal from an originating component being processed (e.g., filtered, amplified, modulated, rectified, attenuated, summed, subtracted, etc.) before being received to the component connected thereto.

The term “one or more” means “at least one” and the term “at least one” means “one or more.” The terms “one or more” and “at least one” include “plurality” as a subset.

The term “substantially,” “generally,” or “about” may be used herein to describe disclosed or claimed embodiments. The term “substantially” may modify a value or relative characteristic disclosed or claimed in the present disclosure. In such instances, “substantially” may signify that the value or relative characteristic it modifies is within ±0%, 0.1%, 0.5%, 1%, 2%, 3%, 4%, 5% or 10% of the value or relative characteristic.

The processes, methods, or algorithms disclosed herein can be deliverable to/implemented by a processing device, controller, or computer, which can include any existing programmable electronic control unit or dedicated electronic control unit. Similarly, the processes, methods, or algorithms can be stored as data and instructions executable by a controller or computer in many forms including, but not limited to, information permanently stored on non-writable storage media such as ROM devices and information alterably stored on writeable storage media such as floppy disks, magnetic tapes, CDs, RAM devices, and other magnetic and optical media. The processes, methods, or algorithms can also be implemented in a software executable object. Alternatively, the processes, methods, or algorithms can be embodied in whole or in part using suitable hardware components, such as Application Specific Integrated Circuits (ASICs), Field-Programmable Gate Arrays (FPGAs), state machines, controllers or other hardware components or devices, or a combination of hardware, software and firmware components.

The term “optimization” means the technical process of determining or selecting a best or improved element or configuration for the cilia described below from a set of available alternatives with regard to some specified criteria (e.g., an objective function, and possibly constraints), and generally within some specified tolerance. In practical use, an optimized system of cilia is improved (with respect to specified criteria), but may or may not be the absolute best or ideal solution. In a refinement, optimization operates to improve a cilia system, and may approach the mathematically optimum solution to within some tolerance (e.g., within 1%, 2%, 5%, or 10% of the mathematically optimal solution). Therefore, the terms “optimized”, “optimum”, and “optimal” mean “improved with respect to specified criteria” (e.g., random or non-optimal selection of parameters).

Throughout this application, where publications are referenced, the disclosures of these publications in their entireties are hereby incorporated by reference into this application to more fully describe the state of the art to which this invention pertains.

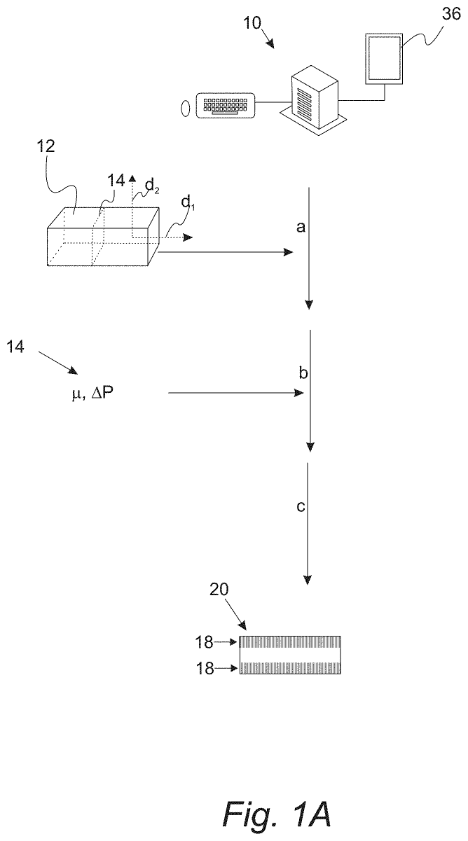

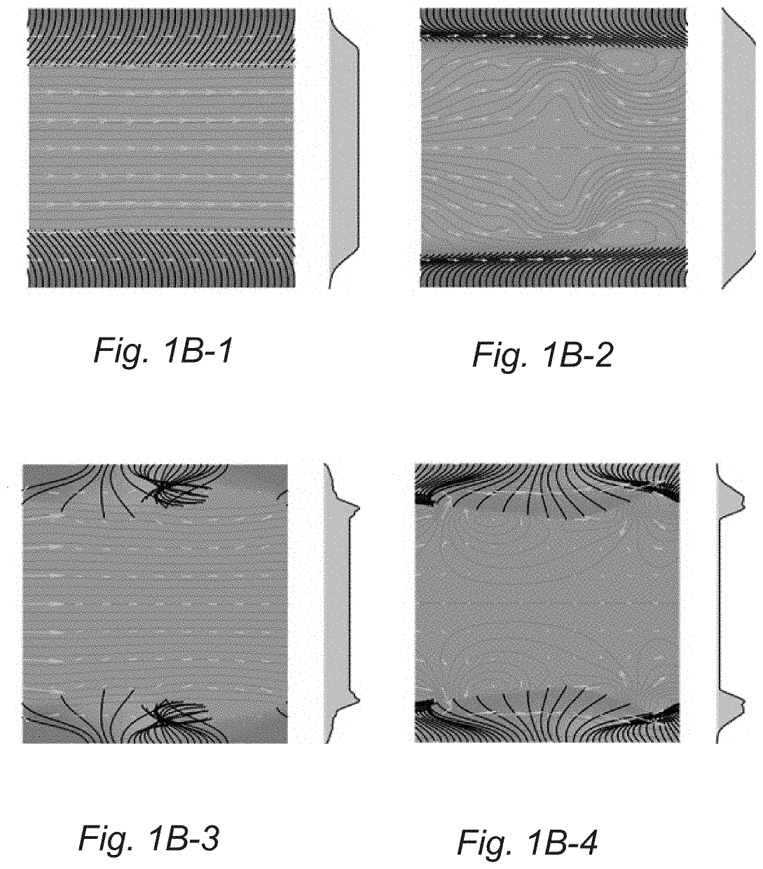

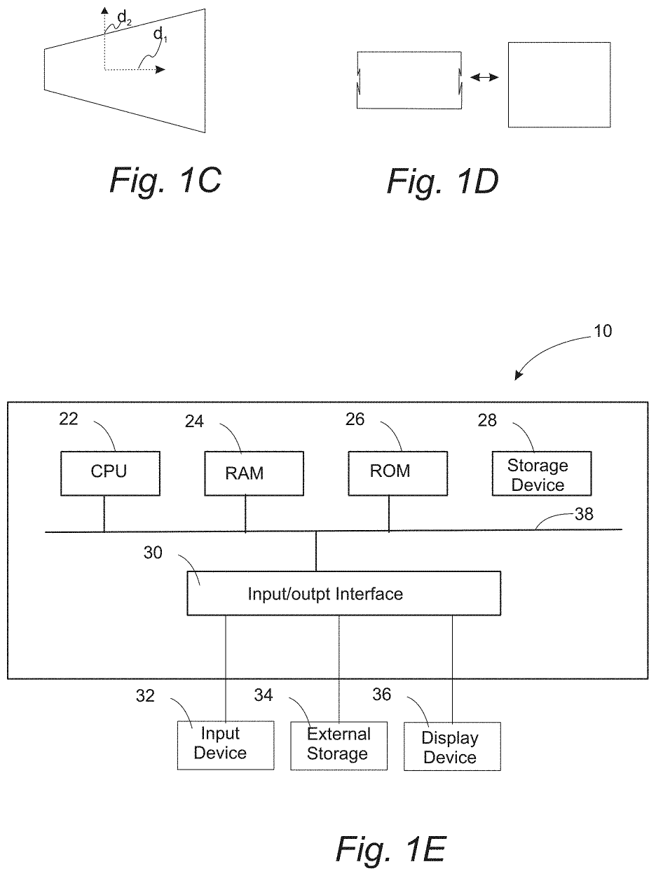

Referring to FIG. 1A, a schematic flow chart depicting a method for designing a microfluidic device is provided. In step a), computing device 10 receives an input design of a bare microfluidic channel 12 to which one or more cilia layers are to be added. Bare microfluidic channel 12 has a predetermined cross section 14 while defining inner surface and an outer surface. First direction d₁ is a fluid flow direction along a length of the bare microfluidic channel while second direction d₂ is perpendicular to the first direction. In step b), computing device 10 receives operation parameters for a ciliated microfluidic channel formed from the bare microfluidic channel. Typically, the operation parameters are parameters that define the environment and conditions in which a cilia or plurality of cilia operate. The operation parameters can include fluid viscosity and an opposing pressure gradient in an adverse direction to the fluid flow direction. In step c), computing device 10 determines cilia design parameters for the one or more cilia layers 18 to be attached to and distributed over the inner surface to form ciliated microfluidic channel 20. Advantageously the cilia design parameters are determined from the incompressible Brinkman equation through optimization. Typically, the cilia design parameters are parameters defining phyical propertires of the cilia or a plurality of cilia. Examples of the cilia design parameters include, but are not limited to cilia density, cilia height, cilia width, and cilia spatial distribution. Additional examples of parameters that can be optimized includes power efficiency I and its defining parameters, an active porous medium of density ρ_{c}, the lumen diameter H, the ciliated fraction h/H, and the combined height of cilia across the duct lumen h as described below. Any one of the parameters described (including the cilia design parameters and the operation parameters) can be used in the optimization. Moreover, any combination of the the parameters described (including the cilia design parameters and the operation parameters) can be used in the optimization.

In a variation, the motion of cilia is a traveling wave. In a refinement, the motion of cilia in the one or more cilia layers is modeled as a longitudinal wave that travels in the first direction. In a further refinement, the motion of cilia in the one or more cilia layers is modeled such that the longitudinal wave remains in sync along the second direction. For example, the one or more cilia layers can be modeled as a square wave, a sinusoidal wave, and/or a two-dimensional wave of any wave profile.

Typically, the incompressible Brinkman equation is described by equation 1:

−μ∇²u(x,y,t)+∇p(x,y,t)+ζ(u(x,y,t)−ν(x,y,t))=−ΔP/L·eₓ∇·u(x,y,t)=0 (1)

wherein:
μ is the fluid viscosity;
u(x,y, t) is a fluid velocity field;
p(x,y, t) the pressure field; and
ζ is the Brinkman coefficient that relates permeability to fluid drag forces which can also be a function of position and time (i.e., ζ(x,y,t));
L is the length of the ciliated microfluidic channel being modeled;
ν(x,y, t) is a velocity field representing motion of cilia in the one or more cilia layers;
ΔP/L represents a uniform pressure gradient in the adverse direction to the fluid flow direction; and
eₓ is a unit vector in the flow direction. The optimization can adjust one or more or any combination of these parameter to determine an optimal value for the parameters. In a refinement, periodic boundary conditions are applied in the fluid flow direction and no-slip boundary conditions are applied in the second direction. Typically, the Brinkman coefficient can be estimated from the total drag force inside the ciliated microfluidic channel.

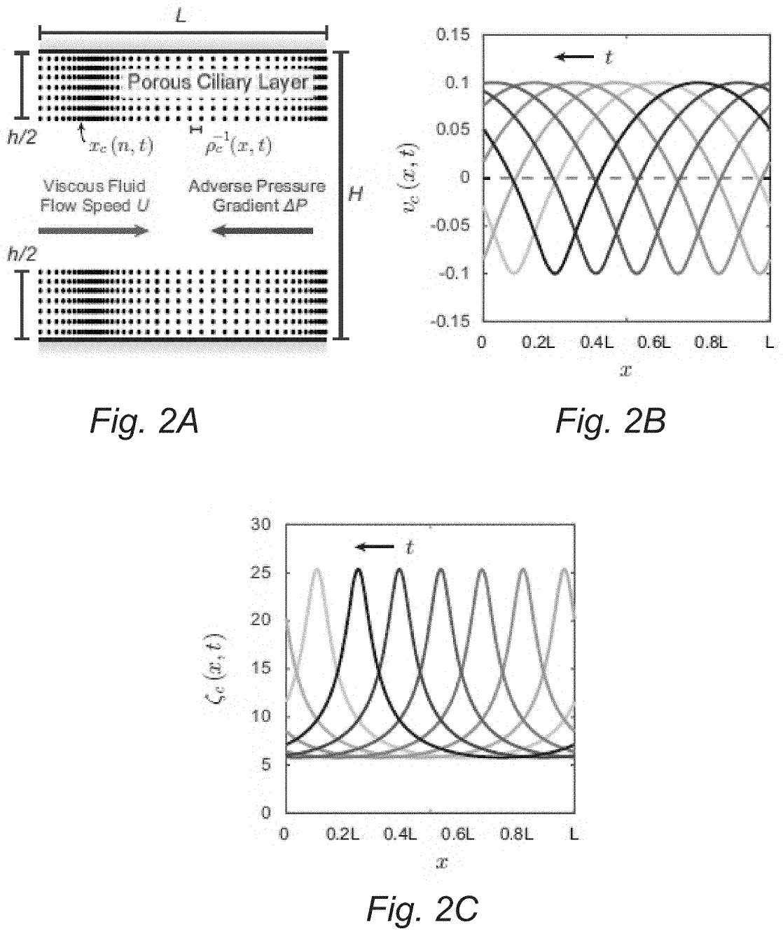

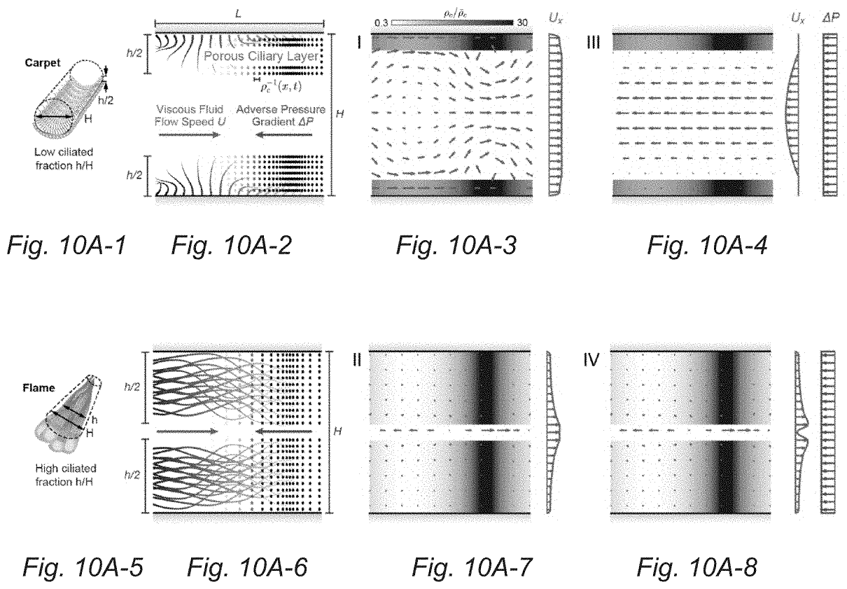

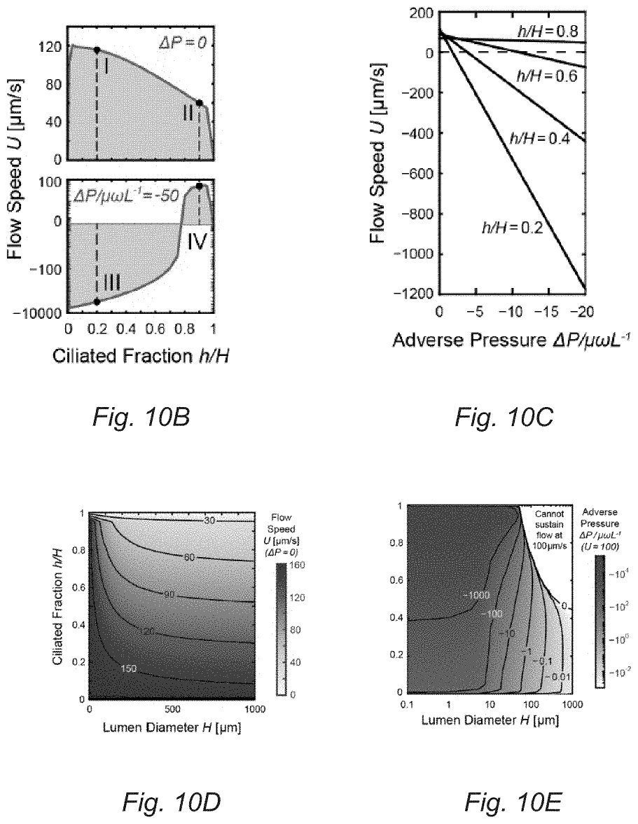

In some examples as depicted in FIG. 1B, cilia that beat synchronously with no metachronal coordination (FIG. 1B-1), cilia that have weak coordination(FIG. 1B-2), cilia whose organization has gaps and with overlap in cilia motion (FIG. 1B-3), and cilia whose beating patterns are taken from biological data such as those base on rabbit tracheal cilia (FIG. 1B-4). In each subfigure, the characteristic cross-sectional flow is shown to the right: the flow is maximized near the cilia and free fluid interface and decays inside the ciliary layer. (similar to what was shown below in FIG. 10A).

In one variation, the microfluidic device is a cilia-driven inline microscale pumps. In another variation, the microfluidic device is an inline microscale filter.

In still another variation, the bare microfluidic channel has a cross-section that varies along the fluid flow direction as depicted in FIG. 1C.

In another aspect, the ciliated microfluidic channel has a cross-sectional area that is adjustable between a first cross-sectional area and a second cross-sectional area as depicted by the cross section in FIG. 1D. In a refinement, a dynamically variable cross section can be form from foldable, swellable, and/or stretchable materials such as hydrogels. Such materials can be molded or eroded/cut into tubes of desired cross-sections that can be tuned for specific elastic and viscous properties, and can be used to embed other living cells, chemicals, structural and fluid/heat/electrically conducting components, as well as sensors, actuators and electronic components.

Characteristically, the bare microfluidic channel is composed of a chemically and biologically inactive material. The chemically and biologically inactive material can include a component selected from the group consisting of acrylics, polysiloxanes, polycarbonates, linear low density polyethylene, acrylonitrile butadiene styrene, a cycloolefin copolymer, glass, mica, silica, semiconductor wafers, and combinations thereof. In a refinement, the chemically and biologically inactive material includes a component selected from the group consisting of collagens, gelatin, hyaluronic acid, chitosan, heparin, alginate, fibrin, polyvinyl alcohol, polyethylene glycol, sodium polyacrylate, acrylate polymers, and copolymers thereof. In another refinement, the bare microfluidic channel is composed of a hydrogel. The microfluidic channel can be fabricated by any number of techniques known in the arts such as micromachining, casting, molding, and the like.

In another variation, the cilia layers include artificial cilia composed of a magnetic material, and/or electrically or thermally driven artificial cilia composed of a smart material such as liquid crystalline elastomers or novel metamaterials. In some variation, the cilia layers can include tissue engineered biological cilia.

In another aspect, a system for designing a microfluidic device is provided. With reference to FIG. 1E, the system includes a computing device includes a processor and memory in electrical communication with the processor. FIG. 1E provides a block diagram of a computing system that can be used to implement the methods set forth above. Computing system 10 includes a processing unit 22 that executes the computer-readable instructions for the computer-implemented steps set forth above. Processing unit 22 can include one or more central processing units (CPU) or microprocessing units (MPU). Computer system 10 also includes RAM 24 or ROM 26 that can have instructions encoded thereon for the steps of the computer implemented method. For example, the at least one computing device 10 can be configured to execute the steps of:

a) receiving an input design of a bare microfluidic channel to which a one or more cilia layers are to be added, the bare microfluidic channel having a predetermined cross section, the bare microfluidic channel defining an inner surface and an outer surface, a first direction being a fluid flow direction along a length of the bare microfluidic channel and a second direction perpendicular to the first direction;

b) receiving operation parameters for a ciliated microfluidic channel formed from the bare microfluidic channel, the operation parameters including fluid viscosity and an opposing pressure gradient in an adverse direction to the fluid flow direction; and

c) determining cilia design parameters for the one or more cilia layers to be attached to and distributed over the inner surface, the cilia design parameters being determined from the incompressible Brinkman equation as set forth above. Typically, the cilia design parameters are determined by an optimization process. For example, the channel diameter H, the ciliated fraction h/H, and the cilia density ρ_{c} can be optimized for maximal flow pumping under a given adverse pressure gradient.

Still referring to FIG. 1E, computer system 10 can also include a secondary storage device 28, such as a hard drive. Input/output interface 300 allows interaction of computing device 10 with an input device 32 such as a keyboard and mouse, external storage 34 (e.g., DVDs and CDROMs), and a display device 36 (e.g., a monitor). Processing unit 102, the RAM 104, the ROM 106, the secondary storage device 28, and input/output interface 30 are in electrical communication with (e.g., connected to) bus 118. During operation, computer system 10 reads computer-executable instructions (e.g., one or more programs) recorded on a non-transitory computer-readable storage medium which can be secondary storage device 28 and or external storage 34. Processing unit 22 executes these reads computer-executable instructions for the computer implemented method set forth above. Specific examples of non-transitory computer-readable storage medium onto which the steps for the computer implemented method are encoded onto include but are not limited to, a hard disk, RAM, ROM, an optical disk (e.g., compact disc, DVD), or Blu-ray Disc (BD)™), a flash memory device, a memory card, and the like.

The following examples illustrate the various embodiments of the present invention. Those skilled in the art will recognize many variations that are within the spirit of the present invention and scope of the claims.

1. Cilia Inspired Pumps

1.1. Brinkman-Stokes Model

Consider a periodic cross-section along the longitudinal x-direction of the ciliated duct, with length L in the x-direction and width H in the orthogonal y-direction. The fluid inside the duct is driven by porous layers of cilia growing from the two side walls of the rectangular channel. The fluid motion is governed by the incompressible Brinkman equation

−μ∇²u+∇p+ζ(u−ν)=−ΔP/L·eₓ,∇·u=0 (1.1)

where μ is the fluid viscosity, u(x,y,t) the fluid velocity field, p(x,y,t) the pressure field, and ζ(x,y,t) the Brinkman coefficient that relates permeability to fluid drag forces. The velocity field v(x,y,t) represents the motion of cilia in the ciliary layers, and ΔP/L on the right hand side represents a uniform pressure gradient in the adverse direction to the desired flow direction. In a refinement, periodic boundary conditions in the longitudinal x-direction and no-slip boundary conditions in the y-direction are applied to the modeling:

u(0,y,t)=u(L,y,t),

p(0,y,t)=p(L,y,t),

u(x,0,t)=u(x,H,t)=0 (1.2).

The minimalist form of activity that pumps fluid in such a channel is to abstract cilia motion into a longitudinal wave that travels in the x-direction but remains in sync along the y-direction; see FIG. 2. In this quasi one-dimensional set up, cilia are only present in the regions y∈(0,h/2] and y∈[H−h/2,H), so it is helpful to define the function y_{c}(y)=1− [symbol] (y−h/2)+ [symbol] (y−H+h/2), where [symbol] is the Heaviside function ( [symbol] (y)=0 for y<0 and 1 otherwise), to decompose the cilia driven velocity field and Brinkman coefficient as,

∇(x,y,t)=ν_{c}(x,t)y_{c}(y)eₓ,

ζ(x,y,t)=ζ_{c}(x,t)y_{c}(y), (1.3)

where ν_{c}(x,t) and ζ_{c}(x,t) are the one-dimensional velocity and Brinkman coefficient due to the longitudinal ciliary waves; see FIG. 2A.

To compute ν_{c}(x,t), let x_{c}(n,t) denote the Lagrangian label of the n^{th} y-column of particles, where n is an integer from 0 to L/b, and b is the average spacing between each particle in the x-direction. Consider that individual particles oscillate with amplitude ϵ and frequency ω, and, with no loss of generality, they coordinate with wavelength that is identical to the channel length L. Thus the Lagrangian description of the particles motion is given by

x_{c}(n,t)=nb+ϵ cos(2πnb/L+ωt). (1.4)

In the continuum limit of nb→x, we get

x_{c}(x,t)=x+ϵ cos(2πx/L+ωt). (1.5)

Information regarding inter-cilium spacing is preserved by defining a uniform packing density of ρ̅_{c}(x,t)=1/b in some reference configuration.

As cilia oscillate, their total number inside the periodic channel is conserved implying that ∫ρ̅_{c}dx=∫ρ_{c}dx_{c}. Therefore, the cilia packing density ρ̅_{c}(x,t) in the current configuration should satisfy

$${ρ}_{c} ( {x}_{c} , t ) = {{\ddot{ρ}}_{c} ( \frac{\partial {x}_{c}}{\partial x} )}^{- 1} = \frac{{\ddot{ρ}}_{c}}{1 - 2 πϵsn ( 2 π x / L + wt ) / L} . \qquad ( 1.6 )$$

Snapshots of ρ_{c}=ρ_{c} is shown in FIGS. 2B and 2C. Similarly, the Lagrangian velocity ν_{c}(x_{c},t) induced by cilia oscillation is

$${v}_{c} ( {x}_{c} , t ) = \frac{\partial {x}_{c}}{\partial t} = - ω ϵ sin ( 2 π x / L + ω t ) . \qquad ( 1.7 )$$

with the understanding that x=x(x_{c},t) on the right-hand side of both (1.6) and (1.7). This inverse map from Lagrangian x_{c} to Eulerian x coordinates can be obtained by inverting (1.5). Since (1.5) is transcendental in x, closed-form expression of this inverse map is not readily available. However, such map always exists whenever (1.6) is non-singular, which is possible as long as ϵ<L/2π. Practically speaking, this inverse map can be approximated either via a series expansion for small ϵ, or through numerical interpolation for finite ϵ. Considering the latter, we calculate x_{c}(x,t) and ν_{c}(x_{c},t) separately in a dense enough grid and interpolate for ν_{c}(x,t) at given x (in MATLAB, we use v=interp1(x_{c}, ν_{c},x)); see FIG. 2B.

Next, we derive an expression for the Brinkman coefficient ζ_{c} (x,t) as follows. We first estimate its value in the reference configuration of stationary equally-spaced particles of radius a that span a distance h/2 in the y-direction (representing the height of the cilia protruding from the duct walls) with horizontal packing density ρ̅_{c}=1/b. Consistent with the Brinkman model, the total drag caused by the motion of all particles is equal to the superposition of drag forces due to each individual particles. Thus the total drag force inside the duct due to particles motion is F_{d}=(ρ̅_{c} L)h/2a)(6πμaν_{c})=3πμρ̅_{c}Lh ν_{c}. Here, ρ̅_{c}L and h/2a represent the number of spheres inside the entire fluid domain in the longitudinal x- and transverse y-directions, respectively. From (1), it is clear that the Brinkman coefficient converts velocity to pressure gradient and has units [(Pa/m)·(m/s)⁻¹)]. In the reference configuration, we have

$${\overline{ζ}}_{c} = \frac{{\overline{ρ}}_{c}}{Lh} \frac{{F}_{d}}{{v}_{c}} = \frac{3 π μ {\overline{ρ}}_{c} Lh}{Lh} \frac{1}{b} = 3 π μ {\overline{ρ}}_{c}^{2} , \qquad ( 1.8 )$$

where we first divide the drag force coefficient by the total ciliated area Lh to get a pressure, and then by the inter-cilium spacing to get a pressure gradient across the mean distance where the drag force is applied. Based on the reference configuration estimate of the Brinkman coefficient, the time varying Brinkman coefficient follows from the conservation law ∫ζ_{c}dx_{c}=∫ζ̅_{c}dx, thus ζ_{c}=ζ̅_{c}(∂x_{c}/∂x)⁻¹, thus ζ_{c}=ζ̅_{c}(∂x_{c}/∂x)⁻¹, giving rise to (FIG. 2C)

ζ_{c}(x,t)=πρ̅_{c}ρ_{c}(x,t). (1.9)

Substituting into (1.3), we arrive at an expression for the Brinkman coefficient (x,y,t).

Lastly, we can also write (1.1) in non-dimensional form by choosing viscosity of the fluid medium μ, periodic duct length L (equivalent to ciliary wavelength), and cilia beat frequency (CBF) m as our characteristic scales as

−∇²ũ+∇_{\tilde{p}}+\tilde{ζ}(ũ−\tilde{ν})=− [symbol] ·\tilde{x} (1.10)

where dimensionless pressure and velocity are defined as

$$\tilde{p} = \frac{p}{ω μ} , \\ \hat{u} = \frac{u}{ω L} , \\ \tilde{v} = \frac{v}{ω L} \qquad ( 1.11 )$$

and \tilde{ζ}=ζL²/μ, and [symbol] =ΔP/(ωμL⁻¹). This leaves us with the following free dimensionless parameters

\tilde{H}=H/L,\tilde{h}=h/L,\tilde{ϵ}=ϵ/L,\overline{\tilde{ρ}}_{c}=ρ̅_{c}L, (1.12)

where \tilde{H} determines the aspect ratio of the channel lumen height to cilia wavelength, \tilde{h} the dimensionless cilia height, \tilde{ϵ} the dimensionless wave amplitude, and \overline{\tilde{ρ}}_{c} the dimensionless cilia density. We drop the (\tilde{·}) notation with the understanding that all quantities are normalized by their respective characteristic scales whenever appropriate. When we need to convert between dimensionless and dimensional quantities, we used by default the viscosity of water at μ=10⁻³ Pa·s, cilia diameter of a=0.125 μm, CBF of ω=15 Hz, and a ciliary wavelength of L=100 μm.

1.2 Pumping Metrics

The effectiveness of a pump is determined by both the quantity of flow it can generate and the efficiency at which it can operate. Since net flow is only possible in x-direction of the channel, we first define the flow profile

$${U}_{x} ( y ) = \frac{1}{T} {\int }_{0}^{T} u ( 0 , y , t ) \cdot {e}_{x} dt = \frac{1}{L} {\int }_{0}^{L} u ( x , y , 0 ) \cdot {e}_{x} dx , \qquad ( 1.13 )$$

where T=2π/ω and the equality follows from the periodicity of the applied forcing and channel boundary conditions in x. The net flow of the channel is thus U= [symbol] Uₓ [symbol] _{y}, where [symbol] · [symbol] _{y} symbolizes averaging along y-direction over channel height H. From this we can define the area flow rate as

Q=UH. (1.14)

Next, we define the efficiency of the channel pump as the power ratio

$$η = \frac{\text{?}}{\text{?}} = \frac{\frac{1}{2} ( \dot{m} \text{?} {U}^{2}}{{〈 {F}_{d} 〉}_{x} {〈 {v}_{c} 〉}_{x}} = \frac{{ρ}_{f} {QU}^{2}}{3 π μ {\overline{ρ}}_{c} L {〈 {v}_{c} 〉}_{x}^{2}} \\ \text{?} \text{indicates text missing or illegible when filed} \qquad ( 1.15 )$$

where m^{·} is the mass flow rate (ρ_{f} r is area density of the fluid) and [symbol] · [symbol] ₓ symbolizes averaging along x-direction of the channel as in eq. (1.13). The average speed [symbol] _{c} [symbol] ₓ of the prescribed traveling wave is given by

$${〈 {v}_{c} ( x , 0 ) 〉}_{x} = \frac{1}{L} {\int }_{0}^{L} {v}_{c} ( x , 0 ) {dx}_{0} = π ω {ϵ}^{2} / L . \qquad ( 1.16 )$$

Put together, the power efficiency η becomes

$$η = \frac{{ρ}_{f} {HU}^{3}}{6 {π}^{2} {ω}^{2} {ϵ}^{4} μ {\overline{ρ}}_{c} {hL}^{- 1}} . \qquad ( 1.17 )$$

This definition of efficiency implies that input power due to ciliary motion is conserved if and only if we keep its denominator as a constant. This hints that to compare pumps with different cilia density and height fairly, ρ̅_{c}h should be constraint to a constant, assuming fixed value for ϵ, ω, μ, and L. In fact, this constraint can also be interpreted as the average amount of cilia material distributed in a unit vertical strip of the channel, and we will keep it as constant when comparing pumps with ciliated fractions and aspect ratios.

1.3 Performance of Ciliary Pumps

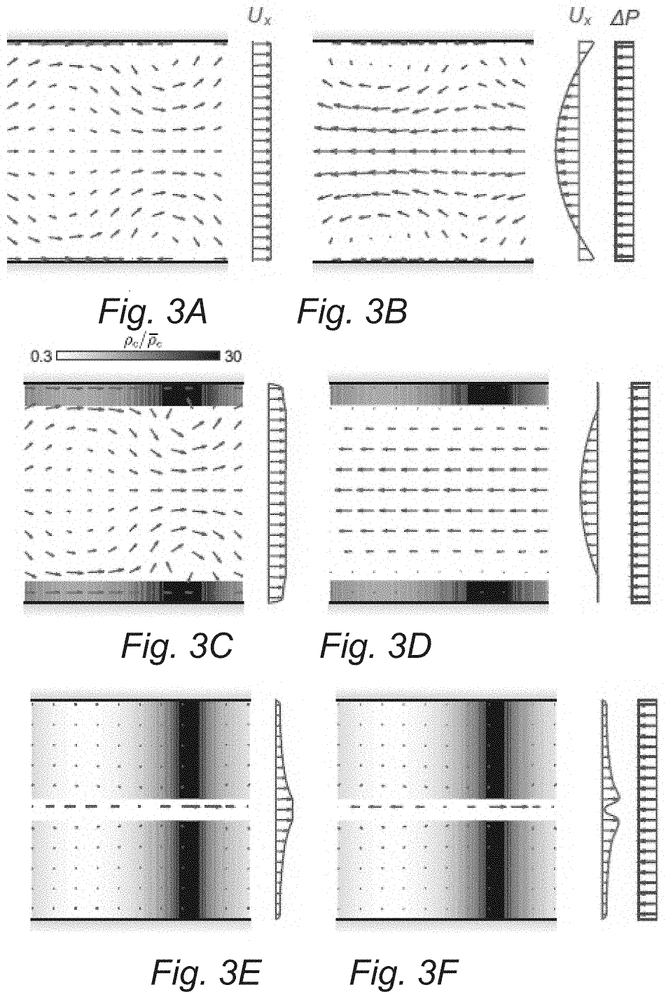

Our porous media model generalizes the classical impermeable sheet model of ciliated surfaces [6]. In the limit of h→0 while keeping ρ̅_{c}h constant, the ciliary layers collapse into infinitesimally thin ciliary carpets that move according to the prescribed longitudinal density wave, such that the governing equation reduces to Stokes flow with slip velocity boundary conditions at the top and bottom walls. This scenario is conceptually equivalent to the study of ciliary flow based on the envelope model [7]. In this limit, mean flow speed U is maximized at the steady state in the absence of adverse pressure since the flow profile Uₓ. can remain constant for all y. However, such pumps are powerless in face of steady adverse pressure; the flow profile reduces to that of Poiseuille's flow with a constant offset; see FIG. 3A-B.

Similarly, if cilia, now represented as active Brinkman layers, remain restricted in height with respect to the channel lumen height (small h/H), mean flow speed U can remain competitive with respect to the theoretical limit proposed by the envelope model in absence of adverse pressure (FIG. 2C). While these pumps can perform better in face of adverse pressure gradient (FIG. 3D), the net flow direction still reverses for large enough |ΔP|. In contrast, when cilia height h approaches lumen height H, pumping performance under no adverse pressure is sacrificed for a continued ability to pump against very large adverse pressure gradient (FIG. 3E-F). This is because while the added adverse pressure forces can easily push the flow in the Stokes flow layer backward, they are relatively ineffective against the active resistance inside the Brinkman layers.

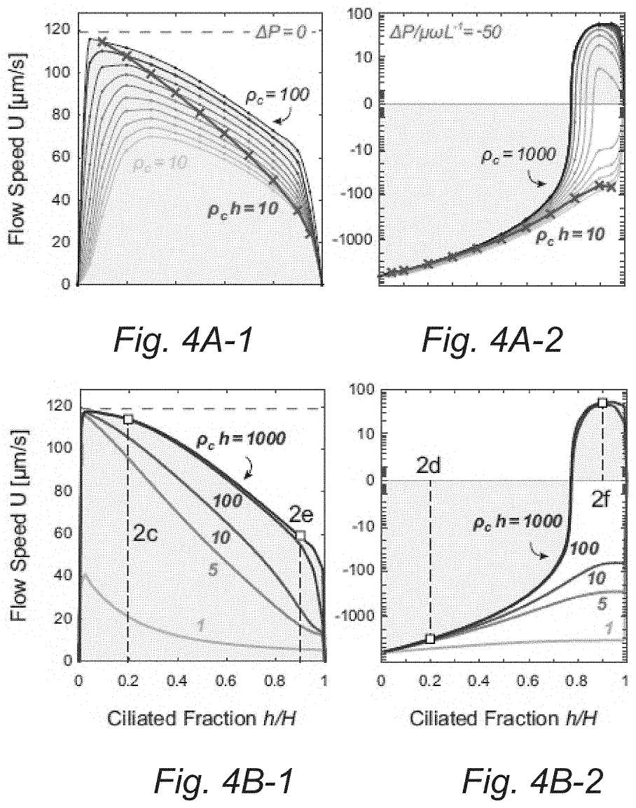

This ability to withstand incoming adverse pressure is mediated by the value of average cilia density ρ̅_{c}h. In FIG. 4A, we show how mean flow speed U changes as a function of ciliated fraction h/H and ρ̅_{c}. On the left panel where adverse pressure is set to 0, we see that the flow speed increases with higher and higher cilia density, but remain bounded above by the analytical speed limit of [symbol] _{c} [symbol] ₓ. In particular, note that even though we increase ρ̅_{c}h by one order of magnitude from 10 to 100, the resultant speed U increments in a much slower fashion. Similarly on the right panel, we show that flow speed again sway towards positive flow direction as ρ̅_{c} increases from 10 to 1000 even in face of an adverse pressure gradient of ΔP/μωL⁻¹=−50. Note that only higher ciliated fraction combined with large enough cilia density can maintain flow in the desired positive direction. However, fully-covered quasi-1D channel do not pump fluids due to continuity constraints, and only act as additional passive resistance when pressure gradients are present; see also App. B. More importantly, if we wish to compare pumps with different ciliated fractions fairly by keeping input power constant, we need to keep the material constraint ρ̅_{c}h as a constant. Shown in blue lines in FIG. 4, we see that for sufficiently large constraint constant, the flow speed performance approaches that of large cilia density. Together these results indicate that there exists a point of diminishing return for high cilia density or material constraint constant.

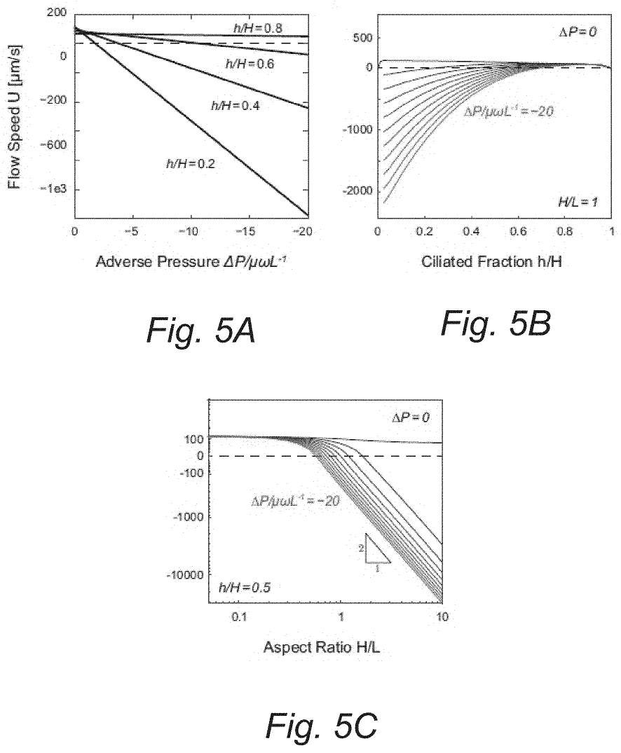

Next we systematically explore the effect of the ciliated fraction h/H, lumen aspect ratio H/L and adverse pressure has on mean flow speed U under the material constraint ρ̅_{c}h=1000. In FIG. 5A, we confirm that Darcy's law applies and mean flow speed is linear with respect to ΔP. However, since U is non-linear in terms of h/H, at larger h/H, U decays more slowly as ΔP increases, hence the ability to produce positive flow at higher adverse pressure. Thus even though larger ciliated fraction is sub-optimal in absence of adverse pressure gradients, they exhibit better performance for sufficiently large ΔP; see FIG. 5B. In addition, as lumen aspect ratio H/L increases, it becomes harder to pump against ΔP as pressure-driven flow is quadratic in H (FIG. 5C). As a result, to construct a ciliary pump that remain functional against very large adverse pressure, both narrow duct openings (relative to wavelength) and high ciliated fraction are required simultaneously. And indeed such trend is evident in the morphospace of (H, h/H) among ciliated pumps observed in biology.

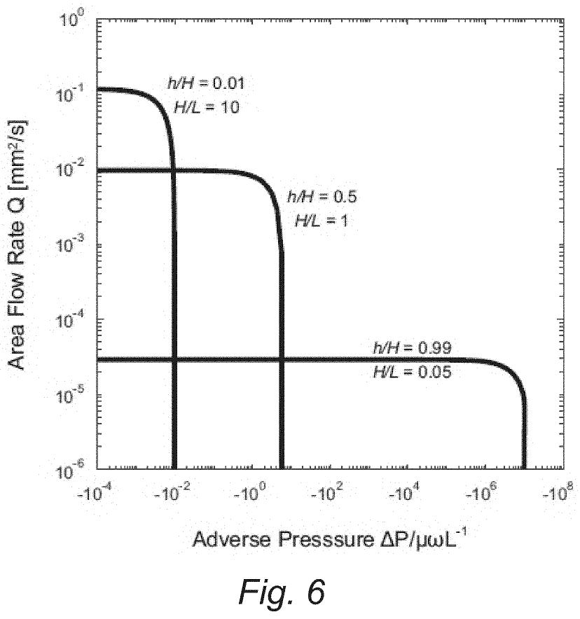

Furthermore, we can demonstrate this design criterion by examining our cilia-inspired pumps in the functional space of flow rate vs. operating pressure, as commonly done in the analysis of engineered pumps (FIG. 6) [9]. Specifically, for a set of fixed geometric parameters such as ciliated fraction, lumen aspect ratio, and material constraint, the pump model produces smaller (area) flow rate Q as adverse pressure ΔP increases in a sharply nonlinear fashion. And to pump at maximum efficiency eta, pumps of all design need to operate near the corner points as seen in FIG. 6.

1.4 Discussion and Conclusion

We presented and analyzed the performance of a cilia-inspired active porous-media pump model. Our results demonstrate mathematically the importance of morphological parameters for ciliary pumps, and has strong implications for the understanding ciliated ducts and tissue in biological settings. We show that permeability is not only natural but necessary for cilia to function as pumps, especially when faced with adverse flow conditions. Specifically, only ciliary pumps with large enough cilia density, high enough ciliated fraction, and small enough lumen size can produce net positive flow against large adverse pressure gradients. In reality, this situation frequently occurs as cilia are responsible for transporting material against forces such as gravity or filter material through highly resistive pathways [10, 11].

On the other hand, insights based on our model are also useful for the design of microfluidic pumps inspired by cilia [12-14. Our results form maps the geometric design parameters that control the distribution of active elements to functional parameters that are useful for performance and cost-benefit analysis. In addition, despite our choice of symmetric beating profile for each Lagrangian particle, this simplification could be desirable for engineered systems.

A few words on generalizations and limitations of our model. In addition to our bulk flow analysis, it would be useful to study particle clearance or residence time in such active porous media framework, as for many ciliated systems, the ultimate goal is to understand how particles embedded in the fluid medium moves so that we can understand the detailed transport and even sensing capability of such pumps. In order to limit the degree of freedom for analysis, we opted for a prescribed, symmetric, and quasi-1D model of cilia activity. It would be straightforward to incorporate asymmetry in beating pattern and two-dimensional traveling waves, or even introduce recorded cilia motion as input to our model. This could also resolve the counter intuitive behavior of full channel stall at (h=H) due to continuity equation. Additionally, generalizing our formulation to an axisymmetric form or one that allows different boundary shapes could also bring additional geometric insight on how ciliated pump perform in more realistic conditions. Moreover, the unsteady term can also be introduced to study the transient behavior of such ciliary pumps and take care of the phase lag that could be introduced due to high frequency of cilia oscillations [16]. And lastly, as hydrodynamic interaction and synchronization between cilia is also of general interest for the study of ciliated tissue, cilia activity can also be introduced such that one can study the two-way coupling between cilia and fluid motion [16].

1.5 Appendix A: Numerical Method

We used a mixed finite element method to solve the non-dimensionalized Brinkman equations by introducing vorticity as an independent variable to avoid numerical evaluation of Laplacian, and solve the fluid equations in weak form on a rectangular mesh. The mixed formulation requires the assignment of discrete vorticity to nodes of the mesh, velocities to edges, and pressure to faces. Correspondingly, bilinear, linear, and piecewise constant basis functions are used for vorticity, velocity and pressure inside each element, respectively; see [17-20]. These compatible discrete basis functions thus allow the incompressibility constraint/continuity equation to be solved simultaneously with the momentum balance equation. Note that in this formulation, the tangential velocity boundary conditions need to be enforced weakly through the vorticity equation, while the normal velocities can be enforced strongly via direct substitution.

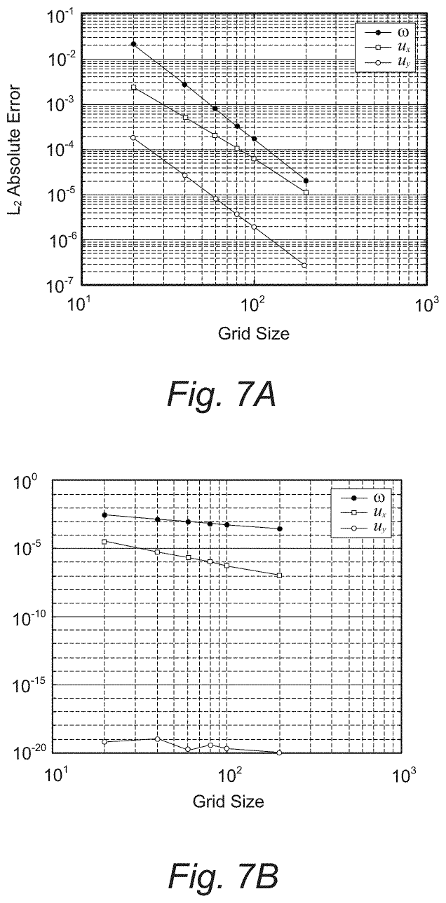

We verified the convergence of this method by first solving two relevant analytically tractable problems: flow inside a periodic channel driven by prescribed boundary flow (envelope model) and pressure-driven Poiseuille flow, see FIGS. 7A and B, respectively. In both cases, we tested with constant non-trivial brinkman coefficients. to ensure pointwise error remain less than O(10⁻⁶)), the duct simulations use mesh sizes no larger than 5×10⁻³ L per side (e.g., 200×200 for H=L and 200×400 for H=2L).

Validation of the Brinkman solver. a. L₂ error convergence of vorticity (black) and velocity (red and blue) for the slip boundary problem, where uₓ(x,0)=uₓ(x,H)=cos(2πx). Tested on unit square domain using grid size of 20×20 to 200×200 with μ=1,L=H=1,ζ=1000. b. Convergence plot for Poiseuille flow with μ=1,L=H=1,ζ=16,ΔP=−1. The error in u_{y} is noisy due to floating point precision (no vertical flow).

As an aside, since the pressure gradient term has only linear effects on the final flow field (FIG. 5A), we can obtain the flow field at any arbitrary ΔP by computing a superposition of a zero adverse pressure flow field and a finite adverse pressure flow field with the necessary weighting, i.e., u(ΔP)=u(ΔP=0)+ΔP/ΔP_{O} [u(ΔPₒ)−u(ΔP=0)].

1.6 Appendix B:Some Analytical Solutions

We derive analytical solutions in two limits: the limit of infinitesimally short ciliary carpet (h→0 and ρ̅_{c}→∞) and the limit of a duct completely filled with our quasi-1D cilia (h=H) in the absence of adverse pressure gradients.

As h→0 and ρ̅_{c}→∞, since we have an expression (1.16) for the average flow generated by ν_{c}, linearity of the Stokes equation implies that, with no adverse pressure gradients, the mean flow speed inside the entire channel would also be exactly that of the prescribed traveling wave πωϵ²L⁻¹. On the other hand, when h=0 and ρ̅_{c} is finite, no cilia is present and the no-slip boundary condition implies no flow can be generated.

If we have h=H, the entire inner duct is subjected to Brinkman flow with no-slip boundaries at the top and bottom. Thus we have v(x,y,t)=ν_{c}(x,t)e\hat{ }ₓ for all γ∈(0,H). It is convenient here to introduce the stream function Ψ such that u=∇×Ψ. The incompressibility constraints is thus satisfied automatically by Ψ, and the governing equation can be rewritten in terms of Ψ by taking curl of (1.1) to arrive at

−μ∇⁴Ψ+ζ∇²Ψ+ζ∇×v=0, (1.18)

subject to the boundary conditions ∇×Ψ=0 at γ=0 and γ=H. Since our choice of longitudinal wave gives ∇×v=0 when h=H, we get that the fluid velocity is identically zero, u=0 everywhere, in absence of adverse pressure gradients. When adverse pressure is present, it reduces to the pressure driven Poiseuille flow problem in 2D periodic channel with an additional resistance contributed by the porous cilia layer, which admits a closed form solution.

2. Flow Physics Explains Morphological Diversity in Ciliated Ducts

2.1 Introduction

Ducts that drive luminal flows by the coordinated beat of internal cilia are ubiquitous in animal biology. In humans, ciliated ducts (CDs) pump fluids in the airways, [21] brain ventricles and spinal canal, [2], and oviduct and efferent ductules of the reproductive system [23, 24]. Their failure is directly linked to major pathologies, including bronchiectasis [25], hydrocephalus [26], and ectopic pregnancy [27]. Cilia in these ducts are short relative to the lumen and oriented perpendicular to the epithelial surfaces, a design commonly referred to as a ciliary carpet [28]. Many invertebrates also feature a strikingly different ciliary arrangement, the so-called ciliary flame [29], where tightly packed long cilia beat longitudinally along a comparatively narrow lumen, reminiscent of a flickering flame. Recent evidence suggests that ciliary flames pump fluid for the purpose of filtration and provide a model system for vertebrate kidney disease [30, 31]. However, despite the fundamental importance of CDs in animal physiology, functional studies and comparisons of intact CDs are rare [24, 32], and the relationship between CD morphology and their ability, or disability, to pump fluid remains largely unexplored.

Existing studies have focused on exposed ciliated surfaces and externally ciliated organisms because of experimental difficulties in measuring ciliary beat and fluid flow inside of intact ducts. The structure, coordination, and microscale flows in planar ciliary carpets can be observed in microdissected ex-vivo epithelia [33] and engineered in-vitro tissues [34, 35], with microfluidic organ-on-chip models emerging as a promising tool to study mucociliary clearance especially in the airways [36, 37]. Additionally, leveraging the remarkable conservation of the ultrastructure of motile cilia among eukaryotes, a wide range of protists, invertebrates, and algae have emerged as powerful model systems for probing the functional spectrum of cilia [38-41]. Insights from these systems combined with mathematical modeling have contributed to a better understanding of the regulation of cilia oscillations by molecular motors [42-46], the coordination between neighboring cilia [41, 47-50], and the role of cilia in mucociliary clearance [33, 51-53], chemosensing [54], locomotion [55], feeding [56-58], symbiotic partnerships between animals and microbes [40, 59], and gas exchange [60, 61].

However, we have yet to relate the architecture of intact CDs to their pumping performance. Establishing this connection could inform our understanding of human ciliopathies [62], aid in comparing different internal fluid transport mechanisms in animals [63, 64], and help in assessing the hypothesized roles of CDs in excretion [30, 31] and host-microbe interactions [65].

In this study, we leverage the unique features of the giant larvacean (Bathochordaeus sp.) model system to study intact CDs. Larvaceans belong to Tunicata (Urochordata), a sister clade of vertebrates, and are an emergent model system for the study of gene regulation, chordate evolution, and development [66]. Bathochordaeus sp. features a multitude of ciliated organs with exceptional optical access [67, 68], providing ideal conditions for studying fundamental properties of cilia-generated flows in intact CDs with direct relevancy to other chordates including humans.

To derive universal structure-function relationships, we combine our functional studies in the giant larvacean with an exhaustive review of published CDs in all animals and physics-based computational models. Specifically, we (i) identify a two-dimensional (2D) morphospace that captures the design of CDs from flames to carpets, (ii) conduct a morphometric analysis of CDs across all animal phyla to identify the distribution of designs in the morphospace, and (iii) explain this design distribution through a unifying physics-based model of the relationship between duct morphology, flow rate, and pressure generation. We arrive at a classification of CD designs in terms of their pumping performance. These results provide a previously unavailable functional assessment of CDs based on morphology that could aid in studying their role in human disease as well as inspire engineered applications.

2.2 Results

2.2.1 Ciliary Carpet Versus Flame Duct Designs

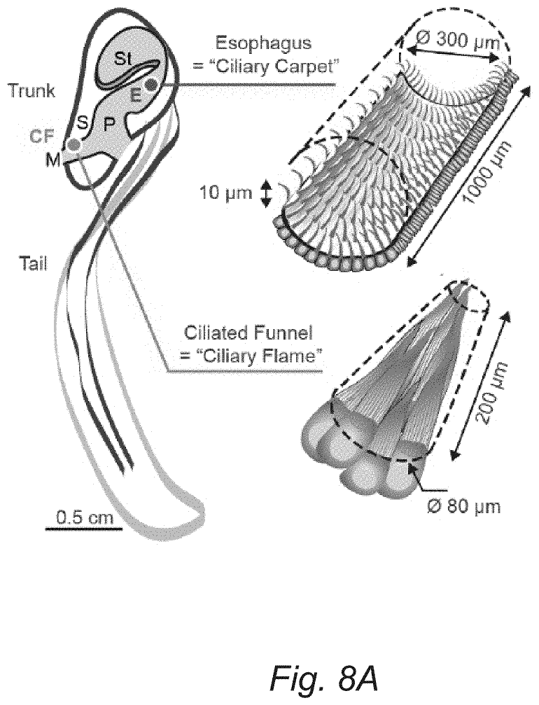

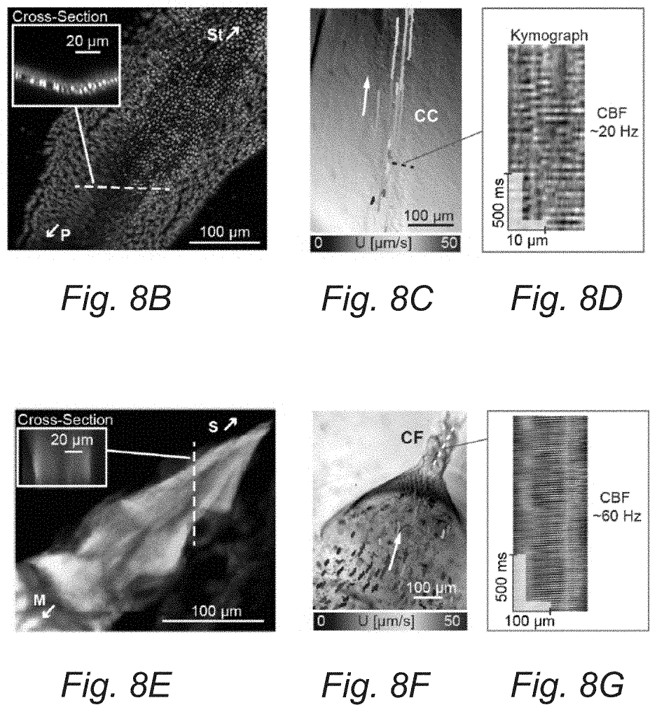

Microscopic inspection revealed that most ciliated ducts in Bathochordaeus sp., such as pharynx, esophagus and gut, exhibit the ciliary carpet design associated with mucus clearance and fluid circulation in humans [69] (FIG. 8A). The filtration-associated ciliary flame design was found in the larvaceans' prominent ciliated funnel [70] (FIG. 8A). The differences in carpet and flame structures suggested a role of cilia length and orientation in modulating fluid transport, and we quantified these parameters in the intact animal. In the esophagus, cilia are much shorter than the width of the lumen (ca. 6 μm vs. 100-200 μm, respectively)(FIG. 8B) and they exhibit ciliary beat frequencies (CBF) of 20 Hz with a stroke cycle perpendicular to the duct walls (FIG. 8d). Neighboring cilia coordinate their beat in metachronal waves that propagate longitudinally along the duct, propelling luminal fluid and food particles at flow speeds of up to 50 μm s⁻¹ (FIG. 8C). These ciliary beat and flow parameters are comparable to those reported in engineered or excised planar ciliary carpets, such as mammalian airway and ependymal ciliated epithelia [22, 71].

In contrast, in the larvacean ciliated funnel, an organ of unknown function near the brain ganglion [70], cilia are longer than the width of the duct lumen (ca. 100 μm versus 55 μm, respectively) and align parallel to the lumen in a densely packed fashion (FIG. 8E). This design—a duct with comparatively small lumen diameter and high ciliated fraction, and with cilia lengths substantially exceeding the lumen diameter—is typical of a ciliary flame [29, 31]. While the beat kinematics of most ciliary flame systems remain unknown, data from our analysis of the ciliated funnel suggest that periodic waves travel along the length of individual cilia, consistent with observations in the flame cells of flatworms [72]. Importantly, the coordinated motion of many parallel cilia generates a longitudinal wave pattern that is reminiscent of the metachronal pattern in ciliary carpets. In the larvacean, the ciliary flame beats at CBFs of up to 60 Hz (FIG. 8G), which falls into the range observed in ciliary carpets [73], and generates equally comparable flow speeds of ca. 40 μm s¹ inside the ciliated duct (estimated from flow towards duct, FIG. 8F).

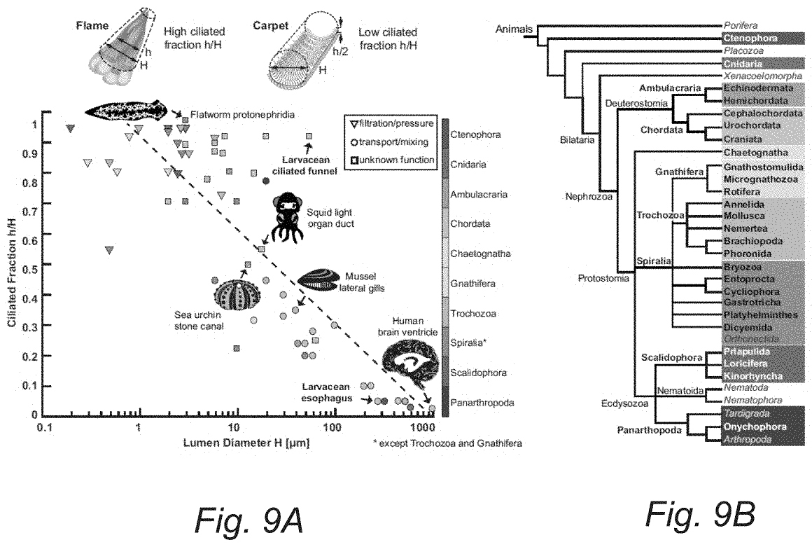

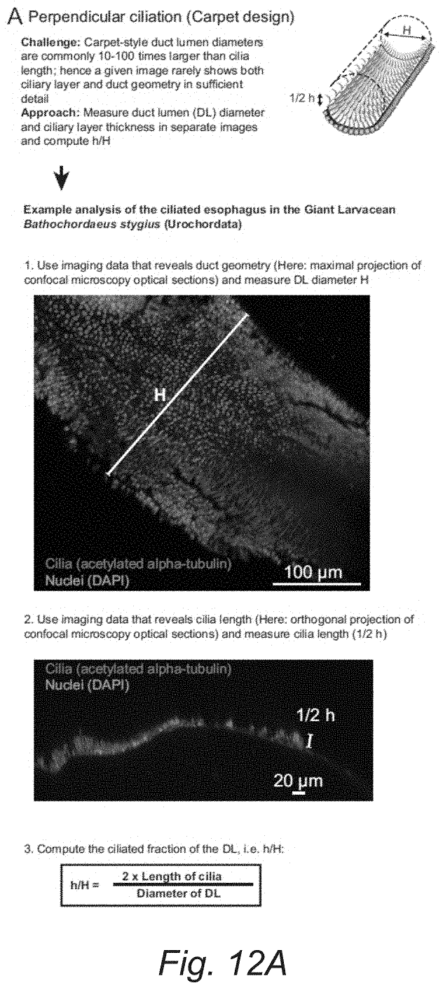

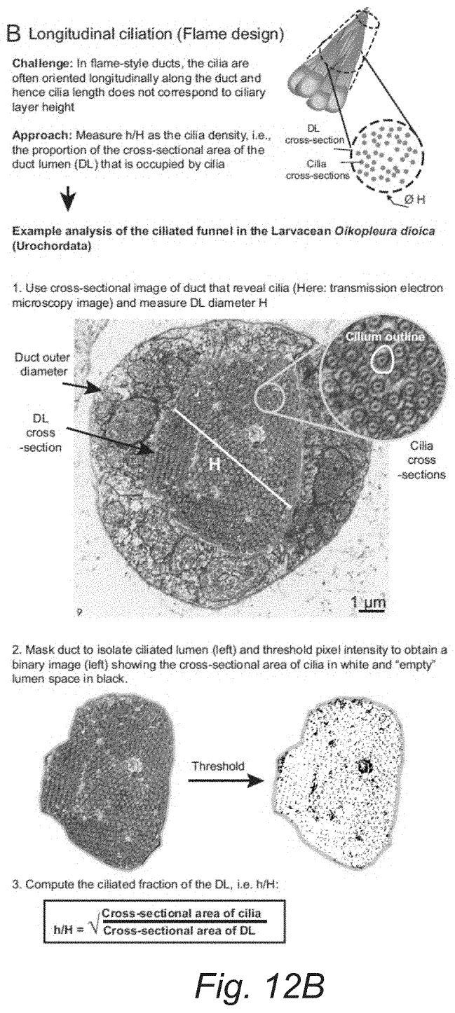

These observations suggested that the lumen diameter H and ciliated fraction h/H, where h is the combined height of cilia across the duct lumen (FIG. 12), are key parameters for characterizing and contrasting the morphologies of CDs in nature (FIG. 9A, top). Importantly, both ciliary carpet and flame ducts appear to generate unidirectional flows (FIGS. 8C-D and F-G) by employing the same active fluid transport mechanism, namely, the use of coordinated ciliary waves traveling along the duct.

2.2.2 Morphometric Analysis Suggests Functional Constraints on Duct Designs

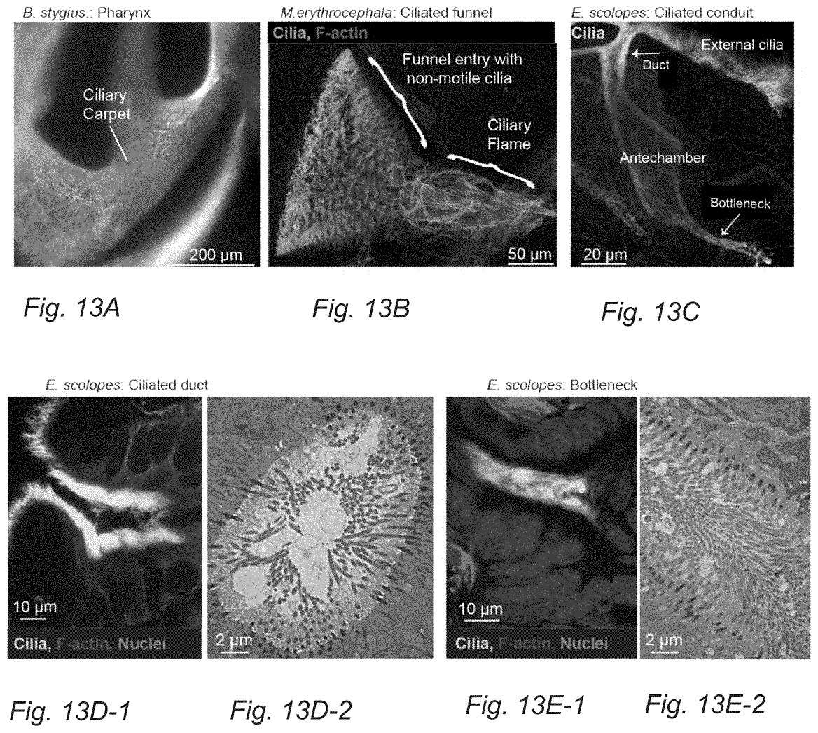

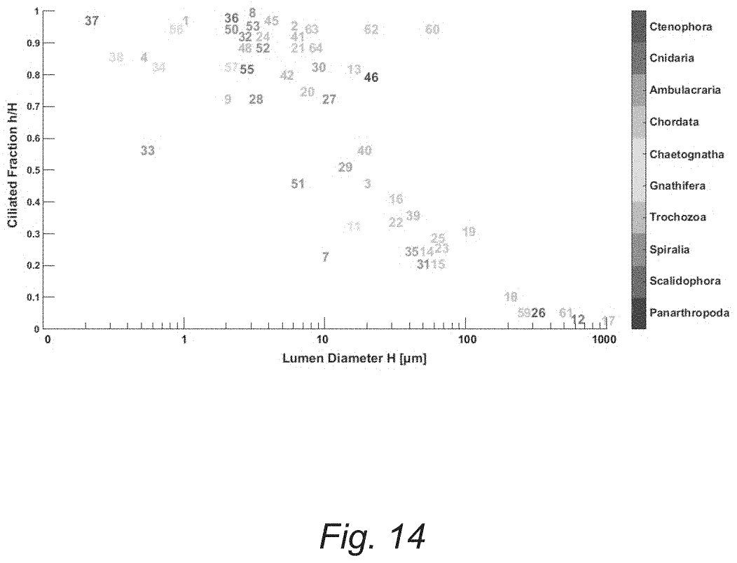

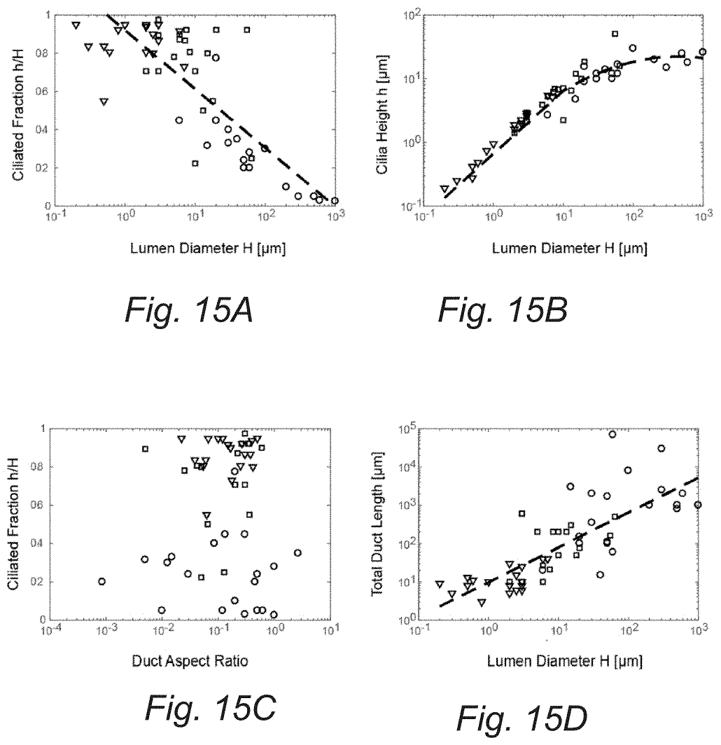

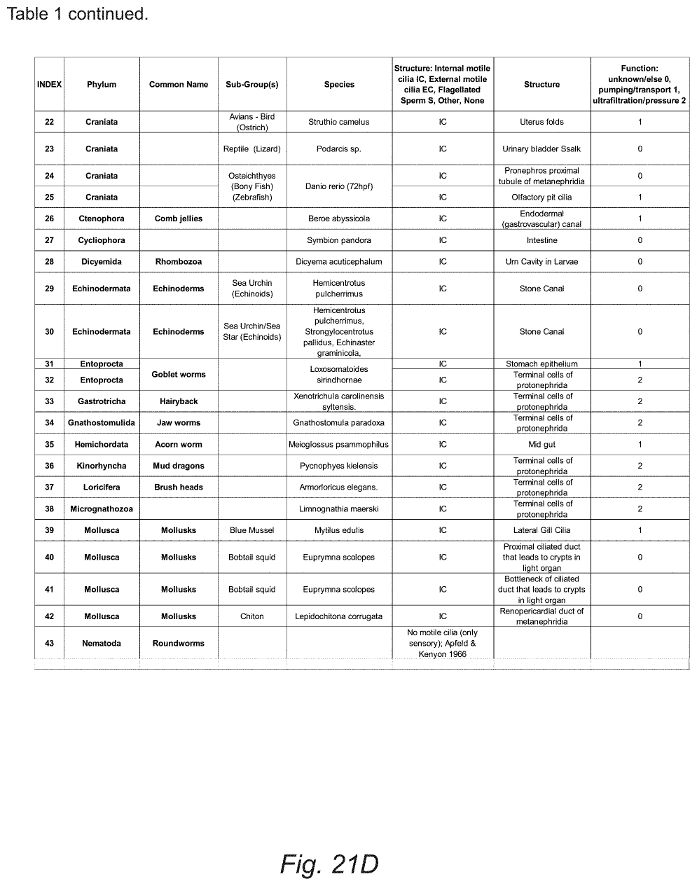

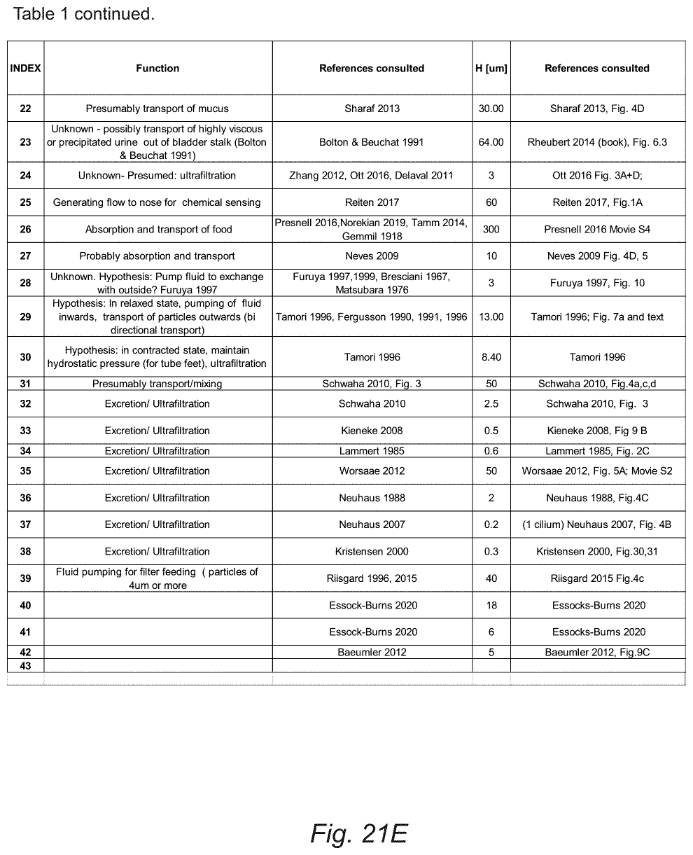

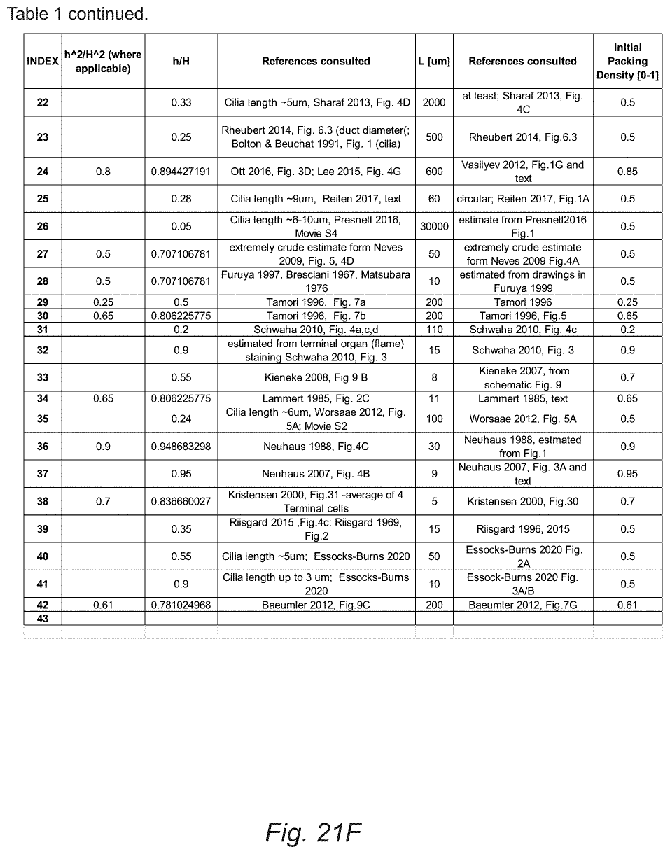

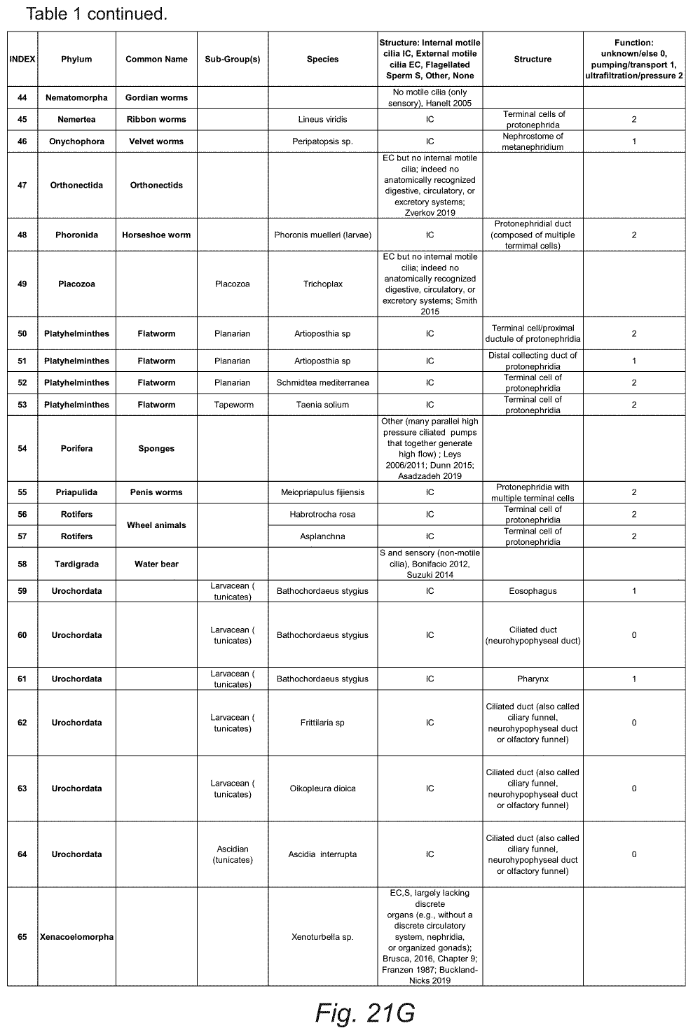

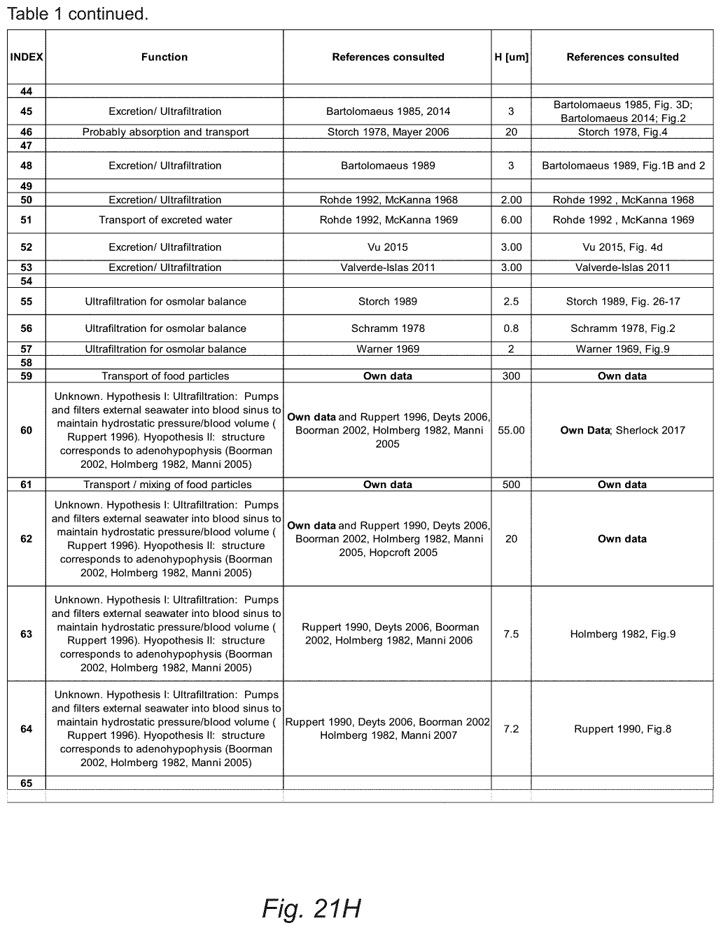

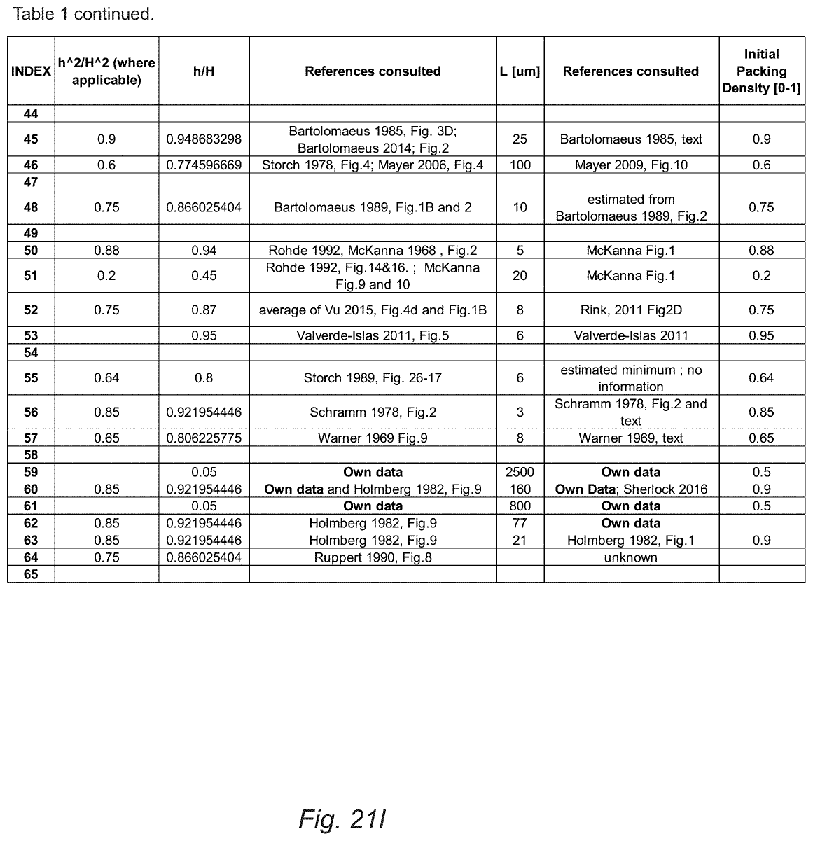

To assess the broader significance of our findings in the chordate model Bathochordaeus sp., we probed the presence and morphology of CDs in all 34 animal phyla [74]. We aimed to test the relevancy of lumen diameter H and ciliated fraction h/H for describing variable duct morphologies, and to evaluate whether phylogenetic distance might be a factor in determining duct morphology. We collected morphometric data (FIG. 12) from published work and our own imaging analysis of Urochordates and Mollusks (FIG. 13), covering a total of 58 duct systems representing 26 animal phyla (FIG. 14 and Table 1, FIG. 21). The remaining 8 phyla either lacked evidence of motile cilia or CDs or, in the case of sponges (phylum Porifera), were excluded from further analysis because of their complex duct morphologies [76, 77]. We observed distinctive variation in the lumen diameter H and ciliated fraction h/H while other structural parameters, such as duct length relative to diameter, lacked obvious differentiating features (FIG. 15). These findings support our hypothesis that CD designs can be described by their unique combination of h/H and H. Plotting h/H versus H for all 58 duct systems showed a well-defined 2D morphospace with a strong correlation between H and h/H (FIG. 9A, bottom). High ciliated fractions (flames) tended to associate with narrow ducts, and low ciliated fractions (carpets) with wider ducts, whereas systems with large H and large h/H, for example, were markedly absent. Mapping known biological functions onto the morphometric distribution showed a clear association of flame designs with filtration, and carpet designs with high-volume fluid transport and mixing. As the distribution of systems in the morphospace was not reflected by their phylogenetic distance (FIGS. 9A and B; and FIG. 14), this suggested a convergent evolution of CD designs based on function.

Intuitively, the design of CDs for fluid pumping is constrained by the metabolic cost of building and actuating the cilia [78, 79], limiting the number and length of cilia on a given ciliated surface. A higher ciliated fraction h/H necessarily also implies less space in the duct lumen that can be taken up by fluid. Minimizing cilia cost while maximizing fluid volume seems to favor a ciliary carpet design at large lumen diameter. However, the ciliary flame designs observed in purported filtration systems suggest that other functional considerations are at play. Specifically, structural data suggest that flame cells achieve filtration by pumping fluid through a flow-resisting sieve [29, 80, 81], indicating that flame designs may be specialized to pump fluid in the presence of high resistance to flow, or, equivalently, adverse pressure gradients that cause counter-currents relative to the desired flow direction. We hypothesized that carpet designs, that transport fluids at comparatively greater lumen diameters and flow rates, require low or zero adverse pressures, whereas flame designs are limited to small diameters and low flow rates because of the high density of active ciliary material required to overcome adverse pressure gradients. These trade-offs would explain both the non-uniform distribution of CD designs and their functional classification.

2.2.3 A Unified Model of Fluid Transport in Carpet and Flame Duct Designs

To rigorously probe this hypothesis, we developed a computational model that compares fluid transport in CDs with different combinations of diameter H and ciliated fraction h/H and evaluate their performance in terms of flow rates, adverse pressure, and overall pumping efficiency [32, 82].

Our model utilizes the Brinkman-Stokes equations and is rooted in that the carpet and flame designs, though morphologically distinct, both operate in a viscous fluid [83, 84] and employ the same pumping mechanism based on active ciliary waves traveling longitudinally along the duct. Specifically, we represented the ciliated fraction by an active porous medium of density ρ_{c} (number of cilia per unit duct length) inside a flow channel (FIG. 10A, Methods § 7 and [85]), with a prescribed spatiotemporal velocity field ν(x,t) that reflects unidirectional ciliary waves. These waves drive the luminal flow field u(x,t) in the CD, including flow that permeates the porous ciliated fractions. The well-known impermeable envelope model for ciliary carpets can be recovered in the limit of small ciliated fraction h/H→0 and high cilia density; see § B.5. The proposed model bridges the two CD designs, from carpets to flames, in a concise way which is unachievable using existing envelope [33, 36, 66-69] or discrete cilia models [48, 53, 58, 90-94], and allows the efficient exploration of the entire parameters space spanned by h/H and H, including designs not found in nature.

2.2.4 Higher Ciliated Fractions Enable Pumping Against Larger Adverse Pressure

We simulated luminal flows u(x,t) using as input the cilia density ρ_{c} (with uniform counterpart ρ_{c} in an undeformed reference configuration) and unidirectional ciliary waves v(x,t) of constant dimensionless amplitude a, wavelength L, and frequency ω. We considered two systems representing the ciliary carpet (h/H=0.2) and flame (h/H=0.9) designs with fixed lumen diameter H=100 μm. The ciliary carpet design generated higher flow speeds than the ciliary flame when no adverse pressure was present (FIG. 10A I and II). In the presence of adverse pressure ΔP, the carpet design failed and allowed reversal of luminal flow, whereas net positive luminal transport remained robust in the flame design (FIG. 10A III and IV).

We quantified the average flow speed U for all ciliated fractions h/H at constant H=100 μm (FIG. 10B). At zero adverse pressure, net positive flows were generated by all ciliated fractions h/H, except in the two limiting cases h/H→0 and h/H→1 (Methods § B.5). Maximal flow speeds on the order of 100 μm s⁻¹ were achieved at small h/H, consistent with experimentally observed flow speeds in ciliary carpets, such as in airway epithelia [71, 95]. However, the flame design was more effective in maintaining positive flow speeds under adverse pressure for a wide range of ΔP values (FIG. 10C).

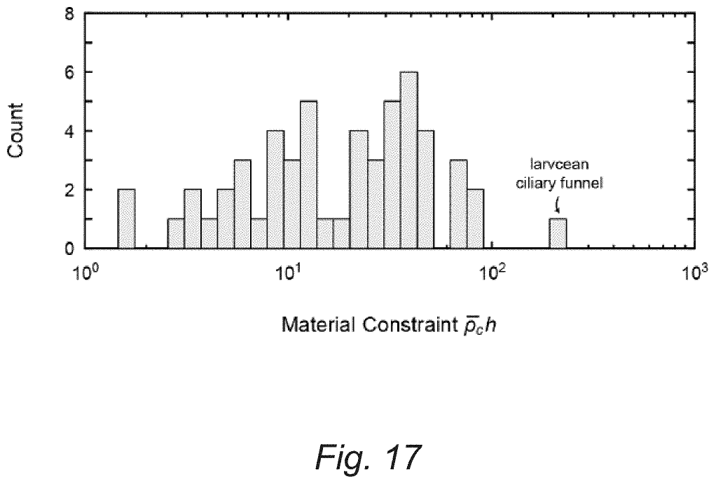

To compare the pumping performance of CDs of similar cilia building and actuation cost, we constrained the total ciliary material in the duct to be constant, which corresponded to conservation of power input to the flow. This constraint is equivalent to setting ρ_{c}h to a constant (Methods § B). We set ρ_{c}h=1000 as an upper bound of all surveyed CDs (FIG. 17) and we systematically explored the flow speeds U over the entire design morphospace (H, h/H). At zero adverse pressure, net positive flows were achievable for any duct design (FIG. 10D). For a desired flow speed, say U=100 μm s⁻¹, viable designs followed a line of solutions connecting carpets (low h/H, high H) to flames (high h/H, low H). However, requiring a net flow speed of U=100 μm s⁻¹ under adverse pressure led to significant changes to the design parameter space: viable designs shifted from encompassing the ciliary carpet design at zero adverse pressure to exclusively requiring the ciliary flame design at large adverse pressures (FIG. 7E). This shift agrees with the hypothesized filtration function of ciliary flames which involves fluid transport in the presence of adverse pressures [30].

2.2.5 Optimal Ducts Shift from Carpet to Flame Designs Under Adverse Pressure

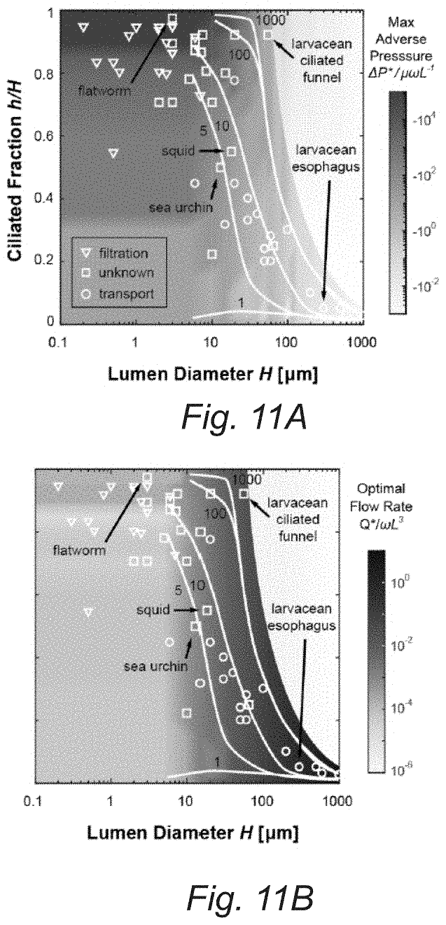

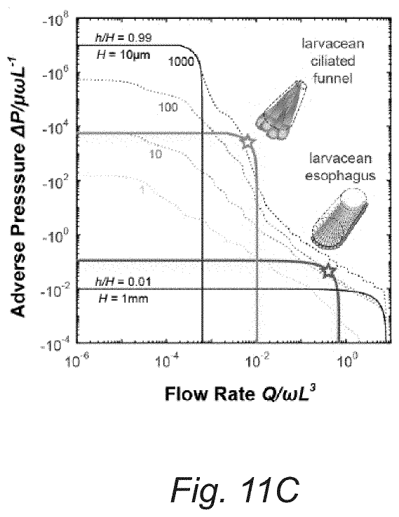

We introduced a pumping efficiency f defined as the ratio of the rate of change of the fluid mechanical energy to the power input by the ciliary layers (Methods § B.4). We maximized efficiency over the three-dimensional space (H, h/H, ΔP) for fixed power input (ρ̅_{c}h=constant). We repeated the same process for different values of ρ̅_{c}h from 1 to 1000 to reflect the entire range found in our morphometric survey (FIG. 17). At ρ̅_{c}h=1000, optimal designs (H, h/H), shown by the solid white line labeled 1000 in FIG. 11A, exhibited a shift from ciliary carpet to ciliary flame designs at larger values of adverse pressure (orange colormap). Optimal designs at lower material constants ρ̅_{c}h corresponded to narrower ducts and lower ciliated fractions. We calculated a volume flow rate Q=(UH² for optimal (H, h/H, ΔP). Note the inverse relationship between the optimal flow rate (green colormap in FIG. 11B) and ability to pump against pressure; carpet designs with large lumen diameter H and small ciliated fraction h/H generate higher volume flows but only function against small adverse pressure gradients, while flame designs with small lumen diameter H and high ciliated fraction h/H can pump fluids against larger values of adverse pressure gradient.

This trade-off between flow rate and pressure is apparent in the function space (Q, ΔP) (FIG. 11C): a ciliary pump (H, h/H) operates along a characteristic curve connecting its maximal generated flow rate at no adverse pressure to the maximal adverse pressure sustained without causing flow reversal. Each ciliary pump functions along its own characteristic curve, depicted in solid lines in FIG. 11C for representative carpet and flame designs, including those of the larvacean shown in FIG. 8. A ciliary pump (H, h/H) is most efficient when operating near the transition from maximal flow to maximal pressure (depicted as stars for the larvacean). Results from our optimization routine (dotted lines) are located at these transitions. The shift from carpet to flame design in the morphospace (H,h/H) (FIG. 11A,B) is accompanied by a shift from maximal flow rate production to maximal pressure generation in the function space (Q, ΔP) (FIG. 11C).

The larvacean funnel curve in FIG. 11C suggests that, when pumping in water with a CBF of ω=60 Hz and ciliary wavelength of L=50 μm, it can provide a maximal flow rate of about 0.2 μl/hr at ΔP≈4 Pa μm⁻¹, which amounts to generating 640 Pa of pressure for the 160 μm long duct. In contrast, a similar calculation estimates that the larvacean esophagus (L=100 μm, ω=20 Hz) can provide a flow of nearly 40 μl hr⁻¹ against a pressure of less than 0.1 Pa over its 2500 μm length.

Lastly, to cast our results in the context of the full range of CD designs observed in nature, we superimposed the biological data from FIG. 9A onto the optimized duct geometries in FIG. 11A,B. Systems whose primary function is to transport luminal fluid reside along the optimized curves in the region of the morphospace with lower ciliated fraction, while systems with presumed filtration functions occupy the region of the morphospace characterized by the ability to sustain higher adverse pressure gradients.

2.3 Discussion

This work establishes a quantitative link between the morphology and pumping function of ciliated ducts. In this light, we now discuss three examples of CDs in FIG. 9A with incomplete functional characterization. For the giant larvecean's ciliated funnel (FIG. 9A), our findings support the decades-old hypothesis that this CD actively maintains the internal volume of the Larvecean seawater-based blood [96] through injection of ultra-filtrated seawater into the blood sinus [97], in addition to the sensory and neuroregulatory functions proposed in contemporary molecular and genetic investigations [70, 98, 99]. We also predict that the ciliated conduit to the light organ of the Hawaian Bobtail Squid (Euprymna socolopes) performs fluid transport functions in its duct and antechamber and filtration or pressurization functions in its bottleneck region (FIG. 13C-E). Additionally, the bottleneck diameter narrows with colonization by bacterial symbionts but widens dramatically during the daily expulsion of bacteria [65], suggesting a shift from a ciliary flame to a ciliary carpet design during this daily venting, with associated changes from filtration to transport function that could aid in maintaining the dynamic host-symbiont association. Similarly, the ciliated pore canals of the madreporite in sea urchins appear to change dynamically between a carpet and flame-like design (Table 1, FIG. 21). Our results support the proposed functional switch between a fluid or particle pump, and a valve that maintains the inner hydrostatic pressure [100].

Our findings may also provide insight into the evolution of waste excretion strategies in the animal kingdom. Our survey shows that filtration by ciliated ducts is limited to animals not exceeding a few milligrams in body mass. Our study provides a potential explanation for its lack in larger organisms. Since cilia have limited length and emanate from the tissue surface, the lumen diameter of a CD cannot increase without eventually lowering the ciliated fraction (note the lack of systems in the large h, large h/H corner of the morphospace). This constraint limits the filtration flow rate that an individual ciliary flame pump can generate. Our model estimates the giant larvacean's ciliated funnel, the largest flame duct in our survey, can sustain a pressure on the order of 640 Pa while pumping at 0.2 μl·hr⁻¹ (FIG. 11C). In contrast, force and metabolism of cardiac muscle, the driver of blood pressure-based filtration, scale much more favorably as a function of muscle volume [101], consistent with the observation that heart mass increases linearly with body mass [102]. The basic vertebrate filtration unit, the glomerulus, operates with blood capillary pressures on the order of 5-10 kPa [83] and has a filtration flow rate of approximately 6 μl·hr⁻¹ [104], exceeding the throughput of the ciliary flame by a factor of more than 30 at more than 10 fold higher pressure. Hence, scaling up cilia-driven filtration to meet equivalent filtration demands would require a disproportionate increase in ciliated surface area by the parallel operation of many pumping units.

Our study also has implications for the functional interpretation of disease phenotypes in CDs [105] and the identification of drugs that disrupt excretory functions of ciliary flame-based CDs in parasites [72]. Our model could inform new flame-like designs for filtration and pressure control in biomimetic and artificially ciliated channels, in addition to existing carpet-like configurations for fluid mixing and transport [106].

Our analysis does have limitations. The survey relied mostly on published data, which is heavily skewed towards popular model systems. Hence, our analysis might be missing alternative CD designs in the “hidden biology” of less studied animals [107]. There are also known ciliated ducts that clearly do not fit in our neat 2D morphospace, such as the ciliated chamber of sponges [76, 77, 78] and some of the unique ciliated cavities found in ctenophores [109-112]. Nevertheless, our 2D morphospace is representative and relevant for most phyla for which evidence of CDs exists. Further, no distinction was made between different types of fluids, such as mucus and water, or between unidirectional and mixing flows. Several systems fall into intermediate ranges of the morphospace, which neither maximize flow rate nor pressure generation. It is possible that these morphologies reflect additional functionalities, such as increased fluid-cilia interaction for chemosensing [113] and mixing [24], or potential transport of chemical signals [22]. We considered a Newtonian fluid model, heuristic cilia waveforms and material constraints, and fundamental measures of pumping performance in order to limit the number of variables and reveal the dominant structure-function relationships of ciliated ducts.

2.4 Methods

A. Data Collection and Measurement Protocol

A.1 Measurement of Duct Lumen Diameter and Ciliated Fraction

Duct lumen (DL) diameter H and ciliated fraction h/H were determined from imaging data (FIGS. 8 and 12) and previously published data (Table 1, FIG. 21). Two different methods were used for quantitative image analysis, depending on the type of ciliation. In carpet-style ciliated ducts (FIG. 12A), where cilia are oriented perpendicular to the duct walls, h/H was determined as the ciliary layer height h divided by DL diameter H. To quantify H, we identified images that showed the duct in its full width or cross-section and directly measured the diameter. To quantify h, we identified images that showed close-ups of the ciliated carpet in cross-sectional or 3D perspective and we measured cilia length. Since ciliary carpets are assumed to line both “floor” and “ceiling” of the ciliated lumen, h corresponds to twice the cilia length. In flame-style ciliated ducts (FIG. 12B), the cilia are aligned longitudinally to the DL and hence cilia density across the channel rather than ciliary length determines the ciliated fraction. h/H was hence determined as the summed cross-sectional area of all cilia divided by the total cross-sectional area of the DL. For this, cross-sectional images of the duct were identified and thresholded to generate a binary image with cilia cross-sections indicated by white pixels and empty lumen by black pixels. Then, h/H was computed by dividing the cilia cross-sectional area (white pixels) by the total cross-sectional area of the DL (black and white pixels) and taking the square root.

In addition to ciliated fraction and lumen diameter, other parameters, including duct length and prescribed ciliary wave length L, were also collected or estimated whenever possible. We have, however, limited confidence in the obtained data on length as pictures and reported morphometric measurements of full-length ducts are rare, and hence our estimates are often based on schematic drawings or text descriptions. Nevertheless, we performed rudimentary data analysis of these data, see FIG. 15.

A.2 Animal Phyla Excluded from the Analysis

As indicated in Table 1 (FIG. 21), phyla were excluded if literature suggested (1) absence of ducts with motile cilia (e.g., Arthropoda, Orthonectida, Placozoa, Tardigrada, Xenacoelomorpha), (2) absence of any motile cilia (e.g., Nematoda, Nematomorpha), or (3) ciliated duct geometries that could not be accurately described with parameters h and H (e.g., Porifera).

A.3 Analysis of Larvacean Sp. (Urochordata)

A3.1 Collections and Husbandry

Different larvacean species (e.g., Bathochordaeus mcnutti, B. stygius, Fritillaria sp.) were collected at water depths ranging from 55 m to 329 m in the Monterey Bay National Marine Sanctuary using acrylic detritus and suction samplers with ROVs Ventana and Doc Ricketts as part of RVs Rachel Carson and Western Flyer cruises in May, October 2016, and June 2018. Collected larvaceans were kept in seawater at 4° C. for a maximum of 48 h before imaging analysis was conducted.

A3.2 Live Imaging and Analysis of Ciliary Beat and Flow

Larvaceans were placed in 10 cm petri dishes filled with seawater at 4° C. that was regularly refreshed. The internal ciliated structures were observed using phase contrast microscopy with 20× and 40× objectives. High-speed video microscopy was performed using either a Sony 4K Handycam FDR-AX700 or a SC1 camera (Edgertronic) mounted each with a custom optical adapter to the c-port or the eyepiece holder of the microscope. Ciliary beat frequency was measured from the videos using the kymograph plugin in ImageJ [114] and measuring the number of beat cycles per second. Flow visualization was achieved by adding 1-10 μm sized glass microspheres (Dantec Dynamics) and polymer microspheres (Cospheric) as tracer particles to the seawater. Fluid flow velocity magnitudes were measured from particle trajectories using the Trackmate plugin in ImageJ [115]. As the high density of cilia in the larvacean ciliated duct obscured the direct imaging of tracer particles, the velocity magnitude within the duct was estimated from the flux in the funnel just outside the duct.

A.3.3 Immunofluorescence (IF) Staining and Imaging of Ciliated Ducts

Animals were fixed in 4 percent paraformaldehyde in seawater for 24 h at 4° C., then washed 3× for 10-30 minutes in phosphate-buffered saline (PBS) and stored in PBS at 4° C. until IF staining. For IF staining of cilia and tissue geometry, the samples were incubated with primary monoclonal antibodies against α-acetylated tubulin (Sigma-Aldrich, St. Louis, Mich., USA) in PBS for 24 h at room temperature, followed by 24 h of incubation in secondary antibody (Invitrogen, Carlsbad, Calif., USA), phalloidin, and DAPI in PBS at room temperature. For IF imaging, tissues were mounted in a custom-made glass-bottom petridish and imaged with a Zeiss LSM 710 laser scanning confocal microscope using a 40× or 63× objective.

A.4 Analysis of Euprymna scolopes (Mollusks)

Animals were cultured and samples were prepared and imaged using laser scanning confocal microscopy and transmission electron microscopy (TEM) as described previously [65].

B. Brinkman Model of Ciliated Ducts

The Reynolds number Re=ρlU/μ, where l is the cilium length, U the fluid velocity, ρ the fluid density and μ its viscosity, reveals the relative importance of viscous versus inertial forces in governing the fluid behavior. Both ciliary carpets and flames have Reynolds number on the order of ˜10⁻³-10⁻², even using fluid viscosities as low as water. Therefore, in both systems inertial forces are negligible and viscous forces are dominant, and the fluid motion is best described in the context of the incompressible Stokes flow model. Manipulating the surrounding fluid in this drag-dominated microscale is not intuitive. For ciliary carpets, the asymmetric power and recovery stroke and metachronal waves break the time-reversible symmetry of Stokes flow, resulting in net fluid transport and pumping [84]. For ciliary flames, symmetry is broken by traveling waves propagating along the cilia length. Given the similarities in the kinematics of the traveling waves and effective flow regimes in the two ciliary phenotyes, we posit that both carpets and flames can be analyzed using a unified fluid-structure interaction model based on a modification of the Brinkman equations that describe flows in porous media.

Classically, models of ciliary carpets represent the tips of densely packed cilia as an impermeable sheet undergoing transverse and longitudinal traveling waves, without accounting for the details of the oscillatory motions of individual cilia. This description is known as the envelope model, which focuses on calculating the fluid motion generated by this moving impermeable sheet [56, 86-89, 116]. Two-way coupled simulations that account for the mechanics of individual cilia also have a long history of development [40, 53, 58, 90-94, 117]. While it is possible to modify these models to account for the dramatic diversity in cilia beating patterns and complex ciliated duct geometries [48], they suffer from having a large number of parameters to be fit to (often unavailable) experimental data and high computational cost for large numbers of cilia. Recently, [118] proposed a coarse-grained model of active filamentous structures in viscous fluids that account for both the elastic response and driving activity of the filament assemblies. In these models, one is contend with an effective representation of the filaments (without representing individual filaments) as an active porous medium that drives and responds to the surrounding fluid. The two-way coupling between the filaments and the surrounding fluid is described by modifying the Brinkman equations for flows past fixed porous media [119, 120]. Here, since our goal is to relate the morphological parameters of the cilia and ciliated duct to the characteristics of cilia-generated flows inside these ciliated ducts, we derived a simpler coarse-grained model based on one-way coupling between the cilia; we ignore the elastic response of the porous medium to flows and represent its activity by a prescribed traveling wave motion. This yields a tractable minimalist computational framework capable of capturing the essential aspects of both short (carpet) and long (flame) cilia beating inside a duct.

Specifically, consider a periodic cross-section along the longitudinal x-direction of the ciliated duct, with length L in the x-direction and width H in the orthogonal y-direction. The fluid inside the duct is driven by porous layers of cilia growing from the two side walls of the rectangular channel. The fluid motion is governed by the incompressible Brinkman equation

−μ∇²u+∇p30ζ(u−ν)=−ΔP/L·eₓ,∇·u=0, (2.1)

where μ is the fluid viscosity, u(x,y,t) the fluid velocity field, p(x,y,t) the pressure field, and ζ(x,y,t) the Brinkman coefficient that relates permeability to fluid drag forces. The velocity field ν(x,y,t) represents to the velocity of the ciliary layers, and ΔP/L on the right hand side represents a uniform pressure gradient in the adverse direction to the desired flow direction. We let Ω=[0,L]×[0,H] denote the fluid domain and consider periodic boundary conditions in the longitudinal x-direction and no-slip boundary conditions in the y-direction,

u(0,y,t)=u(L,y,t),p(0,y,t)=p(L,y,t),

u(x,0,t)=u(x,H,t)=0 (2.2)

Our goal is to compute flow field u(x,y,t) based on a prescribed velocity field ν(x,y,t) representing the cilia traveling wave motions.

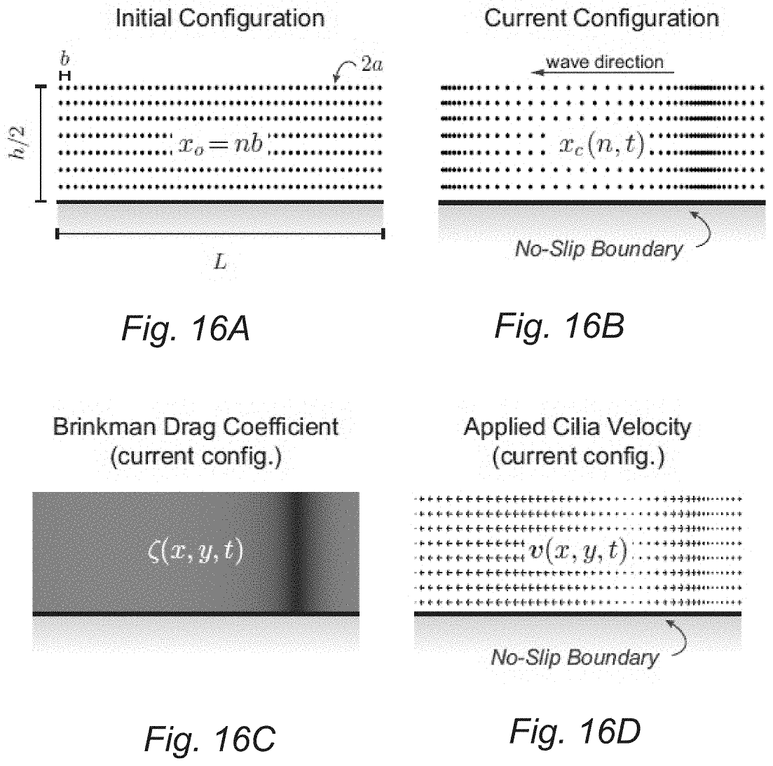

To emulate the two (carpet and flame) designs of ciliated ducts and the spectrum in between, we consider the cilia to protrude a distance h/2 from the duct walls and undergo longitudinal traveling wave motions. Consisting with the Brinkman model, the velocity field v(x,y,t) representing the effect of the cilia activity is derived by modeling the ciliated layers as arrays of equally-spaced particles in the y-direction, with particles in each y-array undergoing the same periodic motion in the x-direction; see FIG. 16.

Given that cilia are only present in the regions y ∈(0, h/2] and y ∈ [H-h/2, H), it is helpful to define the function y_{c}(y)=1− [symbol] (y−h/2)+ [symbol] (y−H+h/2), where [symbol] is the Heaviside function [symbol] [symbol] ( [symbol] )(y)=0 for y<0 and 1 otherwise). The cilia velocity field and Brinkman coefficient in the channel can thus be written as,

ν(x,y,t)=ν_{c}(x,t)y_{c}(y)eₓ,ζ(x,y,t)=ζ_{c}(x,t)y_{c}(y), (2.3)

We next derive expressions for the longitudinal cilia velocity ν_{c}(x,t) and Brinkman coefficient ζ_{c}(x,t) considering longitudinal traveling wave motions in the Brinkman layers.

Let x_{c} denote the Lagrangian label of the n^{th} γ-array of particles, where n is an integer from 0 to N. Here, N the total number of γ-arrays spanning the length L of the duct, and b=L/N the inter-cilium spacing (FIG. 16). Consider that individual particles oscillate with amplitude c and frequency m, and that neighboring particles coordinate in a traveling wave fashion. Further, with no loss of generality, consider that the wavelength of their coordinated motion is identical to the channel length L. We get that the Lagrangian description x_{c}(n, t) of the particles motion is given by

x_{c}(n,t)=nb+ϵ cos(2πnb/L+ωt), (2.4)

In the continuum limit (b→0 and N→∞), nb=nL/N→x, we get

x_{c}(x,t)=x+ϵ cos(2πkx+ωt), (2.5)

where we introduced the wavenumber k=1/L for notational convenience. Information regarding inter-cilium spacing is preserved by defining a uniform packing density of ρ̅_{c}(x,t)=1/b in some reference configuration.

As cilia oscillate, their total number inside the periodic channel is conserved implying that ∫ρ̅_{c}dx=∫ρ_{c}dx_{c}. Therefore, the cilia packing density ρ_{c}(x_{c}, t) in the current configuration should satisfy

$${ρ}_{c} ( {x}_{c} , t ) = {{\overline{ρ}}_{c} ( \frac{\partial {x}_{c}}{\partial x} )}^{- 1} = \frac{{\overline{ρ}}_{c}}{1 - 2 π ϵ k sin ( 2 π k x + ω t )} . \qquad ( 2.6 )$$

Similarly, the Lagrangian velocity ν_{c}(x_{c},t) induced by cilia oscillation is

$${v}_{c} ( {x}_{c} , t ) = \frac{\partial {x}_{c}}{\partial t} = - ω ϵ sin ( 2 π k x + ω t ) . \qquad ( 2.7 )$$

with the understanding that x=x(x_{c}, t) on the right-hand side of both (2.6) and (2.7). This inverse map from Lagrangian x_{c} to Eulerian x coordinates can be obtained by inverting (2.5). Since (2.5) is transcendental in x, closed-form expression of this inverse map is not readily available. However, such map always exists whenever (2.6) is non-singular, which is possible as long as ϵ≤L/2π. Practically speaking, this inverse map can be approximated either via a series expansion for small ϵ, or through numerical interpolation for finite E. Considering the latter, we calculate x_{c}(x, t) and ν_{c}(x_{c}, t) separately in a dense enough grid and interpolate for ν_{c}(x, t) at given x (in MATLAB, we use v=interp1(x_{c}, ν_{c}, x)).

Lastly, we derive an expression for the Brinkman coefficient (x, t) as follows. We first estimate its value in the reference configuration of stationary equally-spaced particles of radius a that span a distance h/2 in the y-direction (representing the height of the cilia protruding from the duct walls) with horizontal packing density ρ̅_{c}=1/b; see leftmost panel of FIG. 16. Consistent with the Brinkman model, the total drag caused by the motion of all particles is equal to the superposition of drag forces due to each individual particles. Thus the total drag force inside the duct due to particles motion is F_{d}(ρ̅_{c}L)h/2α)(6πμaν_{c})=3πμρ̅_{c}Lhν_{c}. Here, ρ̅_{c}L and h/2a represent the number of spheres inside the entire fluid domain in the longitudinal x- and transverse y-directions, respectively. From (2.1), it is clear that the Brinkman coefficient converts velocity to pressure gradient and has units [(Pa/m)·(m/s)⁻¹]. In the reference configuration, we have

$${\overline{ζ}}_{c} = \frac{{\overline{ρ}}_{c}}{Lh} \frac{{F}_{d}}{{c}_{c}} = \frac{3 π μ {\overline{ρ}}_{c} Lh}{Lh} \frac{1}{b} = 3 π μ {\overline{ρ}}_{c}^{2} . \qquad ( 2.8 )$$

where we first divide the drag force coefficient by the total ciliated area Lh to get a pressure, and then by the inter-cilium spacing to get a pressure gradient across the mean distance where the drag force is applied. Based on the reference configuration estimate of the Brinkman coefficient, the time varying Brinkman coefficient follows from the conservation law ∫ζ_{c}dx_{c}=∫ζ̅_{c}dx, thus ζ_{c}=ζ_{c}(∂x_{c}/∂x)⁻¹, giving rise to

ζ_{c}(x,t)=3πμρ̅_{c}ρ_{c}(x,t). (2.9)

Substituting into (2.3), we arrive at an expression for the Brinkman coefficient ζ(x,y,t). Snapshots of the Brinkman coefficient ζ(x,y,t) as well as the corresponding coarse-grained velocity vector field ν(x,y,t) are shown in the two rightmost panels of FIG. 16.

B.1 Linking Brinkman Porosity to Experimental Observations

In the classic 2D Brinkman model, porosity ϕ is defined as the area ratio ϕ=A_{fluid}/Aₜₒₜₐₗ. In our quasi-1D model, we treat the ciliary layer as particles of radius a (taken to be the same as radius of a cilium) packed tightly in the y-direction and with center-to-center distance b in the x-direction in a reference configuration (FIG. 16). This gives us an area porosity ofϕ=1−(h/2a)(πa²)/(hb)=1−(π/2)(a/b). This ϕ is matched with porosity obtained from measurements (FIG. 12) when comparing model and data as detailed below.

Beforehand, we note that by definition the reference density of cilia ρ̅_{c}=1/b, we get the following relationship between the Brinkman porosity ϕ and reference cilia density ρ̅_{c}

$$ϕ = 1 - {\overline{ρ}}_{c} \frac{π a}{2} , \\ or , equivalently , \\ {\overline{ρ}}_{c} = \frac{2 ( 1 - ϕ )}{π a} . \qquad ( 2.1 )$$

This is useful for expressing the ciliary material constraint in terms of ϕ. Indeed, by definition, the material constraint is (ρ̅_{c} L)(h/2a)=constant, which imposes that the total cilia material in the ciliary layer is conserved (hence the total drag force F_{d} generated in the ciliary layer is also fixed). Noting that a and L are held constant in all simulations, we arrive at the equivalent expressions of the material constraint ρ̅_{c} h=constant and

ρ̅_{c}h=constant, or, equivalently,(1−ϕ)h=constant. (2.11)

For ciliary carpets, the fraction of cilia inside the ciliary layer is usually independent to the ciliated fraction h/H. A baseline estimate of ϕ=50% is used to account for both inter-cilium spacing and patchiness of such systems. For CDs with cilia filled throughout the lumen, the measured area ratio of cilia to total lumen (see FIG. 12) can be interpreted both as a direct estimate of the complement of porosity 1-ϕ as well as the dimensionally adjusted ciliated fraction (h/H)². Then using cilium radius a, we can estimate the values of ρ̅_{c} and the material constraint from the above equations; see FIG. 17.

B.2 Non-Dimensionalization and Parameter Value Selection

We write (2.1) in non-dimensional form by choosing viscosity of the fluid medium p, periodic duct length L (which is equal to the ciliary wavelength in computation), and cilia beat frequency (CBF) ω as our characteristic scales. We rewrite the pressure and velocity in dimensionless form as follows

$$\tilde{p} = \frac{p}{ω μ} , \\ \hat{u} = \frac{u}{ω L} , \\ \tilde{v} = \frac{v}{ω L} \qquad ( 2.12 )$$

and the dimensionless parameters

\tilde{H}=H/L,\tilde{h}=h/L,\tilde{ϵ}=ϵ/L=ϵk,\overline{\tilde{ρ}}_{c}L. (2.13)

The non-dimensional Brinkman equation is

−∇²ū+∇\tilde{p}+\tilde{ζ}(ũ−\tilde{ν})=− [symbol] ,eₓ, (2.14)

where \tilde{ζ}=ζL²/μ, and (ΔP)^{\tilde{ }}=ΔP/(ωμL⁻¹). We drop the (\tilde{·}) notation with the understanding that all quantities are normalized by their respective characteristic scales whenever appropriate. When we need to convert between dimensionless and dimensional quantities, we used by default the viscosity of water at μ=10⁻³ Pa·s, cilia diameter of a=0.125 μm, CBF of ω=15 Hz, and a ciliary wavelength of L=100 μm.

To focus our efforts on studying the effects of ciliated fraction h/H, lumen diameter H, and adverse pressure gradient ΔP on the fluid pumping characteristics of CDs, we fix traveling wave amplitude to be ϵ=L/(2π) at its maximal possible value without singularities. The tested numerical range of the remaining parameters are then determined in a manner that respects the order of magnitude based on our experimental observations of the larvacean systems and literature search. Specifically, we tested different ciliated fraction h/H in increments of 5%, examined various lumen diameters in the ranges of 1-1000 μm, and used material constraints ranging from ρ̅_{c}=1 to 1000 (FIG. 17). Since the pressure gradient term has only linear effects on the final flow field (FIG. 10C), we can obtain the flow field at any arbitrary ΔP by computing a superposition of a zero adverse pressure flow field and a finite adverse pressure flow field with the necessary weighting, i.e., u(ΔP)=u(ΔP=0)+ΔP/ΔPₒ [u(ΔPₒ)−u(ΔP=0)].

B.3 CFD Solutions

We used a mixed finite element method to solve the non-dimensionalized Brinkman equations by introducing vorticity as an independent variable to avoid numerical evaluation of Laplacian, and solve the fluid equations in weak form on a rectangular mesh. The mixed formulation requires the assignment of discrete vorticity to nodes of the mesh, velocities to edges, and pressure to faces. Correspondingly, bilinear, linear, and piecewise constant basis functions are used for vorticity, velocity and pressure inside each element, respectively; see [122-125]. These compatible discrete basis functions thus allow the incompressibility constraint/continuity equation to be solved simultaneously with the momentum balance equation. Note that in this formulation, the tangential velocity boundary conditions need to be enforced weakly through the vorticity equation, while the normal velocities can be enforced strongly via direct substitution.

We verified the convergence of this method by first solving two relevant analytically tractable problems: flow inside a periodic channel driven by prescribed boundary flow (akin to the limit case of h→0 and ρ̅_{c}→≈) and pressure-driven Poiseuille flow (see § B.5). In both cases, we tested with constant non-trivial Brinkman coefficients. To ensure pointwise error remain less than O(10⁻⁶), the duct simulations use mesh sizes no larger than 5×10⁻³ L per side (e.g., 200×200 for H=L and 200×400 for H=2L).

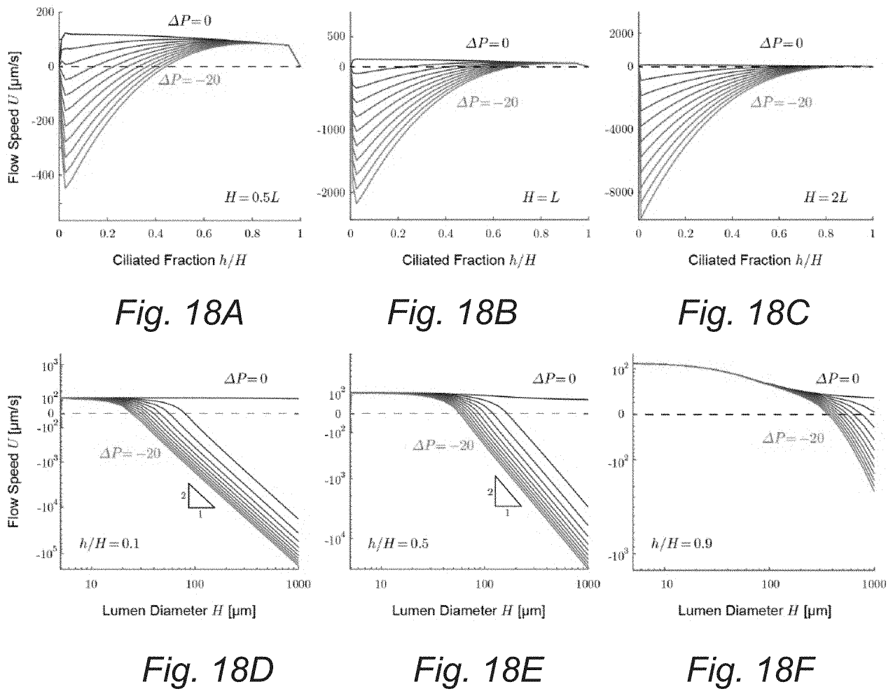

Next, using parameters discussed in § B.2, we found that, in the absence of adverse pressure gradient (ΔP=0), the average flow speed U decreases as h/H and H increase. A small amount of adverse pressure gradient creates background flow for ciliated ducts with small ciliated fraction h/H at large H; see top row of FIG. 18. In fact, once the adverse pressure gradient overwhelms the cilia activity, the backward flow follows a typical Couette flow pattern that is quadratic in lumen diameter H; see bottom row of FIG. 18.

B.4 Optimization Procedures

To obtain the optimal geometric parameters for different pumping scenarios, we use the power efficiency η as an objective function for maximization, Here n is defined as the ratio between output power

$$\frac{1}{2} {〈 \dot{m} 〉}_{x} {〈 u 〉}_{x}^{2}$$

where [symbol] \dot{m} [symbol] ₓ=ρ_{f}UH is the mass flow rate and [symbol] u [symbol] ₓ=U is the average speed, and input power (F_{d})×(ν_{c})ₓ, where (F_{d})ₓ is the average drag force due to ciliary motion and (ν_{c})ₓ is the average speed of the ciliary motion. The brackets (·)ₓ symbolizes averaging over the length direction of the channel, which is equivalent to a temporal cycle-average due to periodicity of the applied forcing and channel boundary conditions in x. The average drag force is (F_{d})ₓ=3πμρ̅_{c}hL(ν_{c})ₓ and the average speed (ν_{c})ₓ of the prescribed traveling wave is given by

$${〈 {v}_{c} 〉}_{x} = \frac{1}{L} {\int }_{0}^{L} {v}_{c} ( x , 0 ) dx = \frac{1}{L} {\int }_{0}^{L} {v}_{c} ( {x}_{c} , 0 ) {dx}_{c} = - π ω {ϵ}^{2} {L}^{- 1} . \qquad ( 2.15 )$$

Put together, the power efficiency η becomes

$$η = \frac{{ρ}_{f} {HU}^{3}}{6 {π}^{2} {ω}^{2} {ϵ}^{4} μ {\overline{ρ}}_{c} {hL}^{- 1}} . \qquad ( 2.16 )$$

Note that by construction, the power input is conserved in all simulations that satisfy the material constraint ρ̅_{c} h=constant, thus, η˜U³H/(ρ_{c}h).

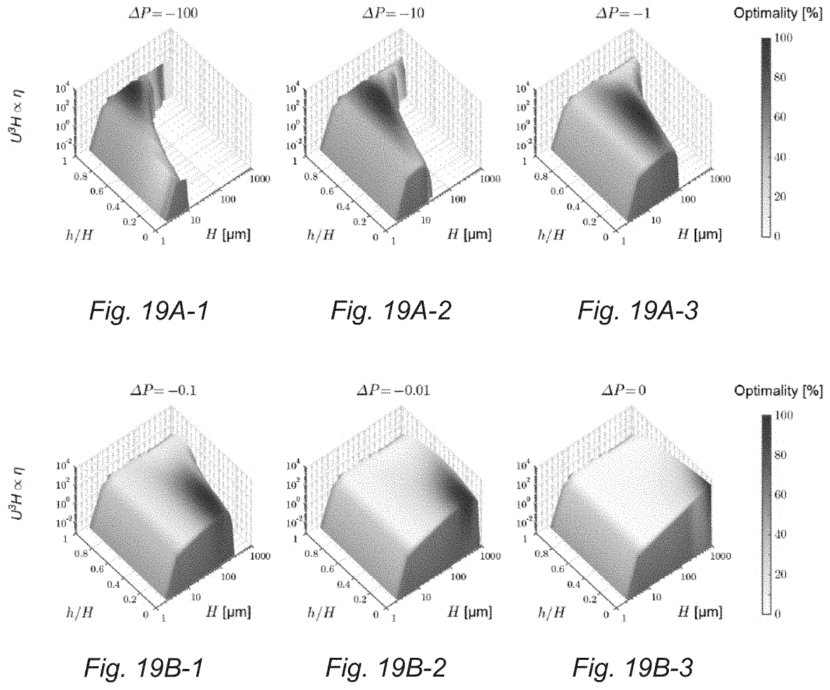

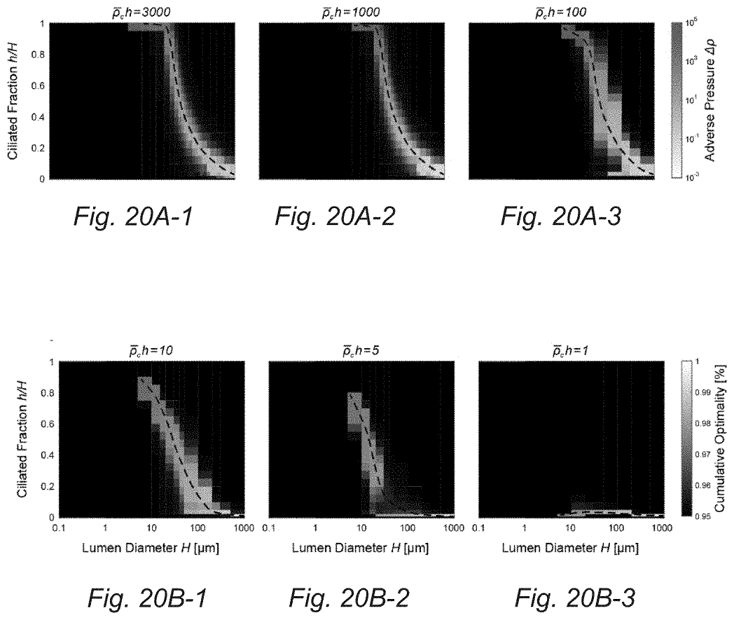

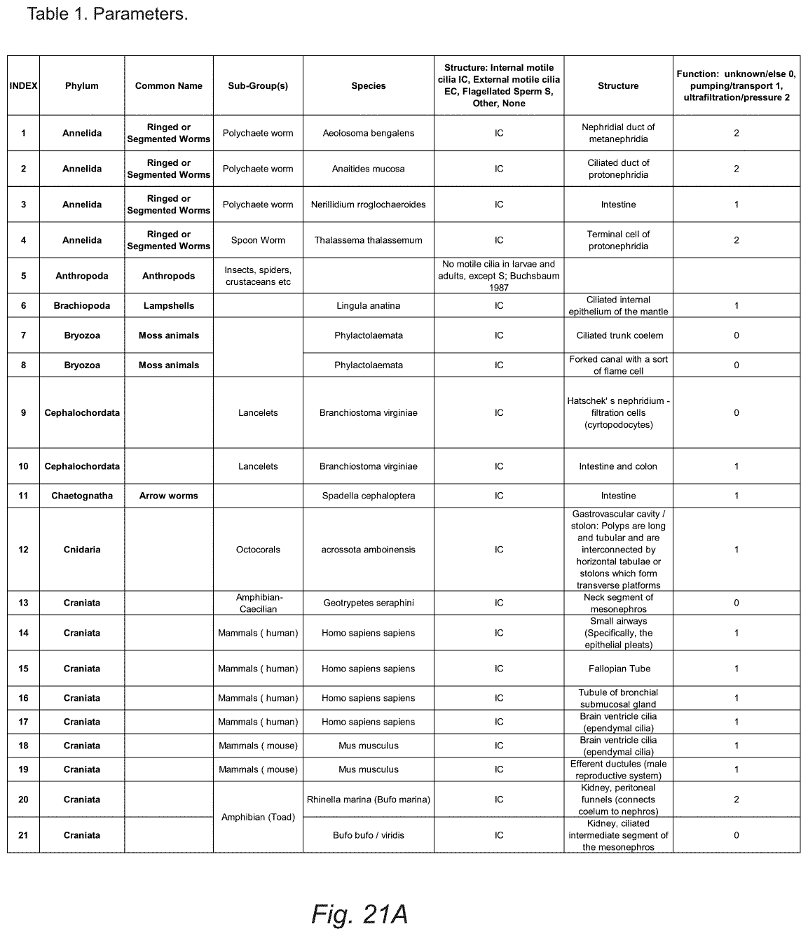

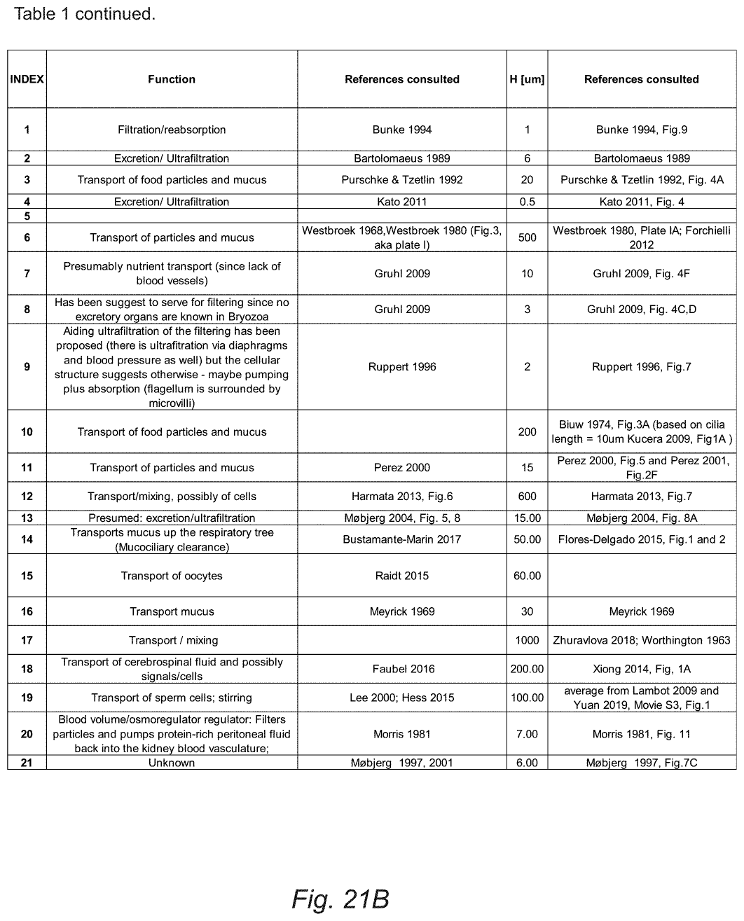

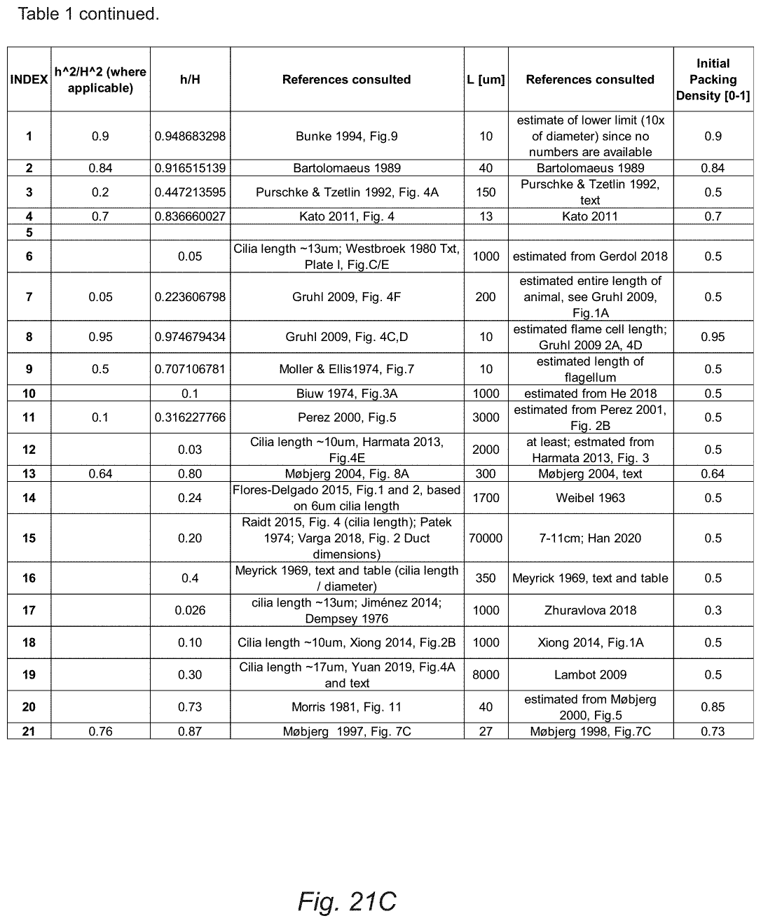

We calculated the maximum efficiency η over the morphological space (H,h/H) for different values of adverse pressure gradients ΔP For each value ΔP, we located the maxima of η dark blue) as shown in FIG. 19, for a given material constraint ρ̅_{c} h=1000. Then, we collected these optimal η values across all ΔP in order to obtain the colormap and optimal dashed line shown in the top left panel of FIG. 20 and the isoline labeled 1000 in FIG. 11A,B of the main text. Namely, the dashed line represents the trace of maximal η values as ΔP varies for the fixed material constraint.

We examined the effect of the value of the material constant ρ̅_{c} h on flow transport. Recall that ρ̅_{c} h represents the amount of active material inside the duct. We calculated optimal η values over the morphological space (H,h/H) as a function of the adverse pressure gradient ΔP for different values of ρ̅_{c} h, see FIG. 20. Note that to highlight the changes near the optimal solutions, we black out part of the parameter space that would produce flow that is less than 95% of their associated optima. The saturation in red indicates the values of ΔP for which forward flow transport is possible. As the constraint ρ̅_{c} h decreases, the cumulative optimality line shift towards the left (smaller and more open channels). Smaller material constraint also reduces the range of adverse pressure gradient for which ciliated ducts can pump against. Dashed lines of FIG. 20 are overlayed next to each other as solid lines in FIG. 11A,B.

B.5 Analytical Solutions of the Brinkman Model in Two Limits

We derive analytical solutions in two limits: the limit of infinitesimally short ciliary carpet (h→0 and ρ̅_{c}→∞) and the limit of a duct completely filled with flame cilia (h=H) in the absence of adverse pressure gradients.

As h→0 and ρ_{c}→∞, the ciliary layers collapse into infinitesimally thin ciliary carpets that move according to the prescribed longitudinal density wave, such that the governing equation reduces to Stokes flow with slip velocity boundary conditions at the top and bottom walls. This scenario is conceptually equivalent to the study of ciliary flow based on the envelope model [87]. Since we have an expression (2.15) for the average flow generated by ν_{c}, linearity of the Stokes equation implies that, with no adverse pressure gradients, the mean flow speed inside the entire channel would also be exactly that of the prescribed traveling wave −πωϵ²L⁻¹. On the other hand, when h=0 and ρ_{c} is finite, no cilia is present and the no-slip boundary condition implies no flow can be generated.

If we have h=H, the entire inner duct is subjected to Brinkman flow with no-slip boundaries at the top and bottom. Thus we have ν(x,y, t)=ν_{c}(x, t)êₓ for all y∈(0,H). It is convenient here to introduce the stream function Ψ such that u=∇×Ψ. The incompressibility constraints is thus satisfied automatically by Ψ, and the governing equation can be rewritten in terms of Ψ by taking curl of (1) to arrive at

−μ∇⁴Ψ+ζ∇²Ψ+ζ∇×ν=0, (2.17

subject to the boundary conditions ∇×Ψ=0 at y=0 and y=H. Since our choice of longitudinal wave gives ∇×v=0 when h=H, we get that the fluid velocity is identically zero, u=0 everywhere, in absence of adverse pressure gradients. When adverse pressure is present, it reduces to the pressure driven Poiseuille flow problem in 2D periodic channel with an additional resistance contributed by the porous cilia layer, which admits a closed form solution.

While exemplary embodiments are described above, it is not intended that these embodiments describe all possible forms of the invention. Rather, the words used in the specification are words of description rather than limitation, and it is understood that various changes may be made without departing from the spirit and scope of the invention. Additionally, the features of various implementing embodiments may be combined to form further embodiments of the invention.

### REFERENCES

[1] Smith C L, Pivovarova N, Reese T S. Coordinated feeding behavior in Trichoplax, an animal without synapses. PLoS One. 2015; 10(9):e0136098.
[2] Nawroth J C, Guo H, Koch E, Heath-Heckman E A, Hermanson J C, Ruby E G, et al. Motile cilia create fluid-mechanical microhabitats for the active recruitment of the host microbiome. Proceedings of the National Academy of Sciences. 2017; 114(36):9510-9516.
[3] Afzelius B A. A human syndrome caused by immotile cilia. Science. 1976; 193(4250):317-319.
[4] Greenstone M, Cole P. Ciliary function in health and disease. British journal of diseases of the chest. 1985; 79:9-26.
[5] Faubel R, Westendorf C, Bodenschatz E, Eichele G. Cilia-based flow network in the brain ventricles. Science. 2016; 353(6295):176-178.
[6] Taylor G I. Analysis of the swimming of microscopic organisms. Proc R Soc Lond A. 1951; 209(1099):447-461.
[7] Blake J. Infinite models for ciliary propulsion. Journal of Fluid Mechanics. 1971; 49(2):209-222.
[8] Webber J B W. A bi-symmetric log transformation for wide-range data. Measurement Science and Technology. 2012; 24(2):027001.
[9] Vogel S. Living in a physical world X. Pumping fluids through conduits. Journal of biosciences. 2007; 32(2):207-222.
[10] Vu H T K, Rink J C, McKinney S A, McClain M, Lakshmanaperumal N, Alexander R, et al. Stem cells and fluid flow drive cyst formation in an invertebrate excretory organ. Elife. 2015; 4:e07405.
[11] McKanna J A. Fine structure of the protonephridial system in Planaria. Zeitschrift fir Zellforschung und Mikroskopische Anatomie. 1968; 92(4):509-523.
[12] Khaderi S N, Craus C, Hussong J, Schorr N, Belardi J, Westerweel J, et al. Magnetically-actuated artificial cilia for microfluidic propulsion. Lab on a Chip. 2011; 11(12):2002-2010.
[13] Hanasoge S, Hesketh P J, Alexeev A. Microfluidic pumping using artificial magnetic cilia. Microsystems & nanoengineering. 2018; 4(1):1-9.
[14] Ul Islam T, Bellouard Y, den Toonder J M. Highly motile nanoscale magnetic artificial cilia. Proceedings of the National Academy of Sciences. 2021; 118(35).
[15] Wei D, Dehnavi P G, Aubin-Tam M E, Tam D. Is the zero Reynolds number approximation valid for ciliary flows? Physical review letters. 2019; 122(12):124502.
[16] Stein D B, Shelley M J. Coarse graining the dynamics of immersed and driven fiber assemblies. Physical Review Fluids. 2019; 4(7):073302.
[17] Bochev P B, Hyman J M. Principles of mimetic discretizations of differential operators. In: Compatible spatial discretizations. Springer, 2006. p. 89-119.
[18] Bochev P, Gunzburger M. A locally conservative mimetic least-squares finite element method for the Stokes equations. In: International Conference on Large-Scale Scientific Computing. Springer; 2009. p. 637-644.
[19] Kreeft J, Gerritsma M. Mixed mimetic spectral element method for Stokes flow: A pointwise divergence-free solution. Journal of Computational Physics. 2013; 240:284-309.
[20] Hiemstra R, Toshniwal D, Huijsmans R, Gerritsma M I. High order geometric methods with exact conservation properties. Journal of Computational Physics. 2014; 257:1444-1471.

### REFERENCES FOR SECTION 2

[21] Bustamante-Marin, X. M. & Ostrowski, L. E. Cilia and mucociliary clearance. Cold Spring Harbor perspectives in biology 9, a028241 (2017).
[22] Faubel, R., Westendorf, C., Bodenschatz, E. & Eichele, G. Cilia-based ow network in the brain ventricles. Science 353, 176-178 (2016).
[23] Raidt, J. et al. Ciliary function and motor protein composition of human fallopian tubes. Human Reproduction 30, 2871-2880 (2015).
[24] Yuan, S. et al. Motile cilia of the male reproductive system require mir-34/mir-449 for development and function to generate luminal turbulence. Proceedings of the National Academy of Sciences 116, 3584-3593 (2019).
[25] Tilley, A. E., Walters, M. S., Shaykhiev, R. & Crystal, R. G. Cilia dysfunction in lung disease. Annual review of physiology 77, 379-406 (2015).
[26] Carter, C. S. et al. Abnormal development of ng2+ pdgfr-+ neural progenitor cells leads to neonatal hydrocephalus in a ciliopathy mouse model. Nature medicine 18, 1797-1804 (2012).
[27] Blyth, M. & Wellesley, D. Ectopic pregnancy in primary ciliary dyskinesia. Journal of Obstetrics and Gynaecology 28, 358-358 (2008).
[28] Gilpin, W., Bull, M. S. & Prakash, M. The multiscale physics of cilia and flagella. Nature Reviews Physics 2, 74-88 (2020).
[29] McKanna, J. A. Fine structure of the protonephridial system in planaria. Zeitschrift f ur Zellforschung und Mikroskopische Anatomie 92, 509-523 (1968).
[30] Rink, J. C., Vu, H. T.-K. & Alvarado, A. S. The maintenance and regeneration of the planarian excretory system are regulated by egfr signaling. Development 138, 3769-3780 (2011).
[31] Vu, H. T.-K. et al. Stem cells and fluid ow drive cyst formation in an invertebrate excretory organ. Elife 4, e07405 (2015).
[32] Vogel, S. Living in a physical world x. pumping fluids through conduits. Journal of biosciences 32, 207-222 (2007).
[33] Ramirez-San Juan, G. R. et al. Multi-scale spatial heterogeneity enhances particle clearance in airway ciliary arrays. Nature Physics 1-7 (2020).
[34] Pellicciotta, N. et al. Cilia density and flow velocity affect alignment of motile cilia from brain cells. Journal of Experimental Biology 223 (2020).
[35] Pellicciotta, N. et al. Entrainment of mammalian motile cilia in the brain with hydrodynamic forces. Proceedings of the National Academy of Sciences 117, 8315-8325 (2020). URL https:/www.pnas.org/content/117/15/8315. https:/www.pnas.org/content/117/15/8315.full.pdf.
[36] Nawroth, J. C. et al. A microengineered airway lung chip models key features of viral-induced exacerbation of asthma. American Journal of Respiratory Cell and Molecular Biology 63, 591-600 (2020).
[37] Sone, N. et al. Multicellular modeling of ciliopathy by combining ips cells and micro uidic airway-on-a-chip technology. Science Translational Medicine 13 (2021).
[38] Short, M. B. et al. Flows driven by flagella of multicellular organisms enhance long-range molecular transport. Proceedings of the National Academy of Sciences 103, 8315-8319 (2006).
[39] Solari, C. A., Ganguly, S., Kessler, J. O., Michod, R. E. & Goldstein, R. E. Multicellularity and the functional interdependence of motility and molecular transport. Proceedings of the National Academy of Sciences 103, 1353-1358 (2006).
[40] Nawroth, J. C. et al. Motile cilia create fluid-mechanical microhabitats for the active recruitment of the host microbiome. Proceedings of the National Academy of Sciences 114, 9510-9516 (2017).
[41] Wan, K. Y. & Goldstein, R. E. Coordinated beating of algal flagella is mediated by basal coupling. Proceedings of the National Academy of Sciences 113, E2784-E2793 (2016).
[42] Julicher, F. & Prost, J. Cooperative molecular motors. Physical review letters 75, 2618 (1995).
[43] Hilfinger, A., Riedel-Kruse, I., Howard, J. & Julicher, F. How molecular motors shape the agellar beat. Biophysical Journal 96, 196a (2009).
[44] Mukundan, V., Sartori, P., Geyer, V., Julicher, F. & Howard, J. Motor regulation results in distal forces that bend partially disintegrated chlamydomonas axonemes into circular arcs. Biophysical journal 106, 2434-2442 (2014).
[45] Ling, F., Guo, H. & Kanso, E. Instability-driven oscillations of elastic microfilaments. Journal of The Royal Society Interface 15, 20180594 (2018).
[46] Man, Y., Ling, F. & Kanso, E. Cilia oscillations. Philosophical Transactions of the Royal Society B 375, 20190157 (2020).
[47] Maestro, A. et al. Control of synchronization in models of hydrodynamically coupled motile cilia. Communications Physics 1, 1-8 (2018).
[48] Guo, H., Zhu, H. & Veerapaneni, S. Simulating cilia-driven mixing and transport in complex geometries. Physical Review Fluids 5, 053103 (2020).
[49] Chakrabarti, B. & Saintillan, D. Hydrodynamic synchronization of spontaneously beating filaments. Physical review letters 123, 208101 (2019).
[50] Man, Y. & Kanso, E. Multisynchrony in active microfilaments. Physical Review Letters 125, 148101 (2020).
[51] Sleigh, M. A., Blake, J. R.& Liron, N. The propulsion of mucus by cilia. American Review of Respiratory Disease 137, 726-741 (1988).
[52] Smith, D. J., Gaffney, E. A. & Blake, J. R. Modelling mucociliary clearance. Respiratory physiology & neurobiology 163, 178-188 (2008).
[53] Ding, Y., Nawroth, J. C., McFall-Ngai, M. J. & Kanso, E. Mixing and transport by ciliary carpets: a numerical study. Journal of Fluid Mechanics 743, 124-140 (2014).
[54] Reiten, I. et al. Motile-cilia-mediated ow improves sensitivity and temporal resolution of olfactory computations. Current Biology 27, 166-174 (2017).
[55] Michelin, S. & Lauga, E. Efficiency optimization and symmetry-breaking in a model of ciliary locomotion. Physics of fluids 22, 111901 (2010).
[56] Michelin, S. & Lauga, E. Optimal feeding is optimal swimming for all peclet numbers. Physics of Fluids 23, 101901 (2011).
[57] Michelin, S. & Lauga, E. Unsteady feeding and optimal strokes of model ciliates. Journal of Fluid Mechanics 715, 1-31 (2013).
[58] Ding, Y. & Kanso, E. Selective particle capture by asynchronously beating cilia. Physics of Fluids 27, 121902 (2015).
[59] Kanso, E. A., Lopes, R. M., Strickler, J. R., Dabiri, J. O. & Costello, J. H. Teamwork in the viscous oceanic microscale. Proceedings of the National Academy of Sciences 118 (2021). URL https://www.pnas.org/content/118/29/e2018193118. https://www.pnas.org/content/118/29/e2018193118.full.pdf.
[60] Gilpin, W., Prakash, V. N. & Prakash, M. Vortex arrays and ciliary tangles underlie the feeding-swimming trade-off in star fish larvae. Nature Physics 13, 380-386 (2017).
[61] Shapiro, O. H. et al. Vortical ciliary flows actively enhance mass transport in reef corals. Proceedings of the National Academy of Sciences 111, 13391-13396 (2014).
[62] Hildebrandt, F., Benzing, T. & Katsanis, N. Ciliopathies. New England Journal of Medicine 364, 1533-1543 (2011).
[63] Andrikou, C., Thiel, D., Ruiz-Santiesteban, J. A. & Hejnol, A. Active mode of excretion across digestive tissues predates the origin of excretory organs. PLoS biology 17, e3000408 (2019).
[64] Ichimura, K. & Sakai, T. Evolutionary morphology of podocytes and primary urine-producing apparatus. Anatomical science international 92, 161-172 (2017).
[65] Essock-Burns, T., Bongrand, C., Goldman, W. E., Ruby, E. G. & McFall-Ngai,
M. J. Interactions of symbiotic partners drive the development of a complex biogeography in the squid-vibrio symbiosis. Mbio 11 (2020).
[66] Glover, J. C. Oikopleura. Current Biology 30, R1243-R1245 (2020).
[67] Sherlock, R., Walz, K., Schlining, K. & Robison, B. Morphology, ecology, and molecular biology of a new species of giant larvacean in the eastern north pacific: Bathochordaeus mcnutti sp. nov. Marine biology 164, 20 (2017).
[68] Katija, K. et al. Revealing enigmatic mucus structures in the deep sea using deeppiv. Nature 583, 78-82 (2020).
[69] Meunier, A. & Azimzadeh, J. Multiciliated cells in animals. Cold Spring Harbor perspectives in biology 8, a028233 (2016).
[70] Holmberg, K. The ciliated brain duct of oikopleura dioica (tunicata, appendicularia). Acta Zoologica 63, 101-109 (1982).
[71] Sears, P. R., Yin, W.-N. & Ostrowski, L. E. Continuous mucociliary transport by primary human airway epithelial cells in vitro. American Journal of Physiology-Lung Cellular and Molecular Physiology 309, L99-L108 (2015).
[72] Valverde-Islas, L. E. et al. Visualization and 3d reconstruction of ame cells of taenia solium (cestoda). PloS one 6, e14754 (2011).
[73] Olstad, E. W. et al. Ciliary beating compartmentalizes cerebrospinal fluid flow in the brain and regulates ventricular development. Current Biology 29, 229-241 (2019).
[74] Dunn, C. W., Giribet, G., Edgecombe, G. D. & Hejnol, A. Animal phylogeny and its evolutionary implications. Annual review of ecology, evolution, and systematics 45, 371-395 (2014).
[75] Feuda, R. et al. Improved modeling of compositional heterogeneity supports sponges as sister to all other animals. Current Biology 27, 3864-3870 (2017).
[76] Asadzadeh, S. S., Larsen, P. S., Riisgard, H. U. & Walther, J. H. Hydrodynamics of the leucon sponge pump. Journal of the Royal Society Interface 16, 20180630 (2019).
[77] Leys, S. P. et al. The sponge pump: the role of current induced flow in the design of the sponge body plan. PloS one 6, e27787 (2011).
[78] Marshall, W. F., Qin, H., Brenni, M. R. & Rosenbaum, J. L. Flagellar length control system: testing a simple model based on intra flagellar transport and turnover. Molecular biology of the cell 16, 270-278 (2005).
[79] Ishikawa, H. & Marshall, W. F. Ciliogenesis: building the cell's antenna. Nature reviews Molecular cell biology 12, 222-234 (2011).
[80] Scimone, M. L., Srivastava, M., Bell, G. W. & Reddien, P. W. A regulatory program for excretory system regeneration in planarians. Development 138, 4387-4398 (2011).
[81] Ruppert, E. E. & Smith, P. R. The functional organization of filtration nephridia. Biological Reviews 63, 231-258 (1988).
[82] Forouhar, A. S. et al. The embryonic vertebrate heart tube is a dynamic suction pump. Science 312, 751-753 (2006).
[83] Purcell, E. M. The efficiency of propulsion by a rotating flagellum. Proceedings of the National Academy of Sciences 94, 11307-11311 (1997).
[84] Lauga, E. & Powers, T. R. The hydrodynamics of swimming microorganisms. Reports on Progress in Physics 72, 096601 (2009).
[85] Ling, F. et al. Cilia inspired pumps. arXiv preprint (2021).
[86] Taylor, G. I. Analysis of the swimming of microscopic organisms. Proceedings of the Royal Society of London A 209, 447-461 (1951).
[87] Blake, J. Infinite models for ciliary propulsion. Journal of Fluid Mechanics 49, 209-222 (1971).
[88] Pak, O. S. & Lauga, E. The transient swimming of a waving sheet. Proceedings of the Royal Society of London A 466, 107-126 (2010).
[89] Chrispell, J. C., Fauci, L. J. & Shelley, M. An actuated elastic sheet interacting with passive and active structures in a viscoelastic fluid. Physics of Fluids 25, e1002167 (2013).
[90] Liron, N. & Mochon, S. The discrete-cilia approach to propulsion of ciliated micro-organisms. Journal of Fluid Mechanics 75, 593-607 (1976).
[91] Liron, N. Fluid transport by cilia between parallel plates. Journal of Fluid Mechanics 86, 705-726 (1978).
[92] Gueron, S. & Liron, N. Ciliary motion modeling, and dynamic multicilia interactions. Biophysical journal 63, 1045-1058 (1992).
[93] Blake, J., Liron, N. & Aldis, G. Flow patterns around ciliated microorganisms and in ciliated ducts. Journal of theoretical biology 98, 127-141 (1982).
[94] Guo, H. & Kanso, E. Evaluating efficiency and robustness in cilia design. Physical Review E 93, 033119 (2016).
[95] Kelly, S. J., Brodecky, V., Skuza, E. M., Berger, P. & Tatkov, S. Variability in tracheal mucociliary transport is not controlled by beating cilia in lambs in vivo during ventilation with humidified and nonhumidified air. American Journal of Physiology-Lung Cellular and Molecular Physiology 320, L473-L485 (2021).
[96] ROBERTSON, J. D. The chemical composition of the blood of some aquatic chordates, including members of the tunicata, cyclostomata and osteichthyes. Journal of Experimental Biology 31, 424-442 (1954).
[97] Ruppert, E. E. Structure, ultrastructure and function of the neural gland complex of ascidia interrupta (chordata, ascidiacea): clarification of hypotheses regarding the evolution of the vertebrate anterior pituitary. Acta Zoologica 71, 135-149 (1990).
[98] Bassham, S. & Postlethwait, J. H. The evolutionary history of placodes: a molecular genetic investigation of the larvacean urochordate oikopleura dioica. Development 132, 4259-4272 (2005).
[99] Deyts, C., Casane, D., Vernier, P., Bourrat, F. & Joly, J.-S. Morphological and gene expression similarities suggest that the ascidian neural gland may be osmoregulatory and homologous to vertebrate peri-ventricular organs. European Journal of Neuroscience 24, 2299-2308 (2006).
[100] Tamori, M., Matsuno, A. & Takahashi, K. Structure and function of the pore canals of the sea urchin madreporite. Philosophical Transactions of the Royal Society of London. Series B: Biological Sciences 351, 659-676 (1996).
[101] Marden, J. H. Scaling of maximum net force output by motors used for locomotion. Journal of Experimental Biology 208, 1653-1664 (2005).
[102] Dawson, T. J. et al. Functional capacities of marsupial hearts: size and mitochondrial parameters indicate higher aerobic capabilities than generally seen in placental mammals. Journal of Comparative Physiology B 173, 583-590 (2003).
[103] Gottschalk, C. W. A review of intrarenal hemodynamics and hydrodynamics. Urodynamics 299-308 (1971).
[104] Rubenstein, D., Yin, W. & Frame, M. D. Bio fluid mechanics: an introduction to fluid mechanics, macrocirculation, and microcirculation (Academic Press, 2015).
[105] Ott, E. et al. Pronephric tubule morphogenesis in zebra fish depends on mnx mediated repression of irxlb within the intermediate mesoderm. Developmental Biology 411, 101-114 (2016).
[106] Chen, C.-Y., Cheng, L.-Y., Hsu, C.-C. & Mani, K. Microscale flow propulsion through bioinspired and magnetically actuated artificial cilia. Biomicrofluidics 9, 034105 (2015).
[107] Dunn, C. W., Leys, S. P. & Haddock, S. H. The hidden biology of sponges and ctenophores. Trends in ecology & evolution 30, 282-291 (2015).
[108] Leys, S. P. & Eerkes-Medrano, D. I. Feeding in a calcareous sponge: particle uptake by pseudopodia. The Biological Bulletin 211, 157-171 (2006).
[109] Norekian, T. P. & Moroz, L. L. Neural system and receptor diversity in the ctenophore beroe abyssicola. Journal of Comparative Neurology 527, 1986-2008 (2019).
[110] Tamm, S. L. Cilia and the life of ctenophores. Invertebrate Biology 133, 1-46 (2014).
[111] Gemmill, J. F. Ciliary action in the internal cavities of the ctenophore pleurobrachia pileus fabr. Proceedings of the Zoological Society of London 88, 263-265 (1918).
[112] Presnell, J. S. et al. The presence of a functionally tripartite through-gut in ctenophora has implications for metazoan character trait evolution. Current Biology 26, 2814-2820 (2016).
[113] Shah, A. S., Ben-Shahar, Y., Moninger, T. O., Kline, J. N. & Welsh, M. J. Motile cilia of human airway epithelia are chemosensory. Science 325, 1131-1134 (2009).
[114] Abramoff, M. D., Magalhaes, P. J. & Ram, S. J. Image processing with imagej. Biophotonics international 11, 36-42 (2004).
[115] Tinevez, J.-Y. et al. Trackmate: An open and extensible platform for single-particle tracking. Methods 115, 80-90 (2017).
[116] Blake, J. A model for the micro-structure in ciliated organisms. Journal of Fluid Mechanics 55, 1-23 (1972).
[117] Guo, H., Nawroth, J., Ding, Y. & Kanso, E. Cilia beating patterns are not hydrodynamically optimal. Physics of Fluids 26, 091901 (2014).
[118] Stein, D. B. & Shelley, M. J. Coarse graining the dynamics of immersed and driven fiber assemblies. Physical Review Fluids 4, 073302 (2019).
[119] Wuttanachamsri, K. & Schreyer, L. Effects of cilia movement on fluid velocity: Ii numerical solutions over a fixed domain. Transport in Porous Media 134, 471-489 (2020).
[120] Wuttanachamsri, K. & Schreyer, L. Effects of cilia movement on fluid velocity: I model of fluid flow due to a moving solid in a porous media framework. Transport in Porous Media 136, 699-714 (2021).
[121] Webber, J. B. W. A bi-symmetric log transformation for wide-range data. Measurement Science and Technology 24, 027001 (2012).
[122] Bochev, P. B. & Hyman, J. M. Principles of mimetic discretizations of differential operators. In Compatible spatial discretizations, 89-119 (Springer, 2006).
[123] Bochev, P. & Gunzburger, M. A locally conservative mimetic least-squares finite element method for the stokes equations. In International Conference on Large-Scale Scientific Computing, 637-644 (Springer, 2009).
[124] Kreeft, J. & Gerritsma, M. Mixed mimetic spectral element method for stokes flow: A pointwise divergence-free solution. Journal of Computational Physics 240, 284-309 (2013).
[125] Hiemstra, R., Toshniwal, D., Huijsmans, R. & Gerritsma, M. I. High order geometric methods with exact conservation properties. Journal of Computational Physics 257, 1444-1471 (2014).

What is claimed is:

1. A method for designing a microfluidic device performed by a computing device, the method comprising:
receiving an input design of a bare microfluidic channel to which one or more cilia layers are to be added, the bare microfluidic channel having a predetermined cross section, the bare microfluidic channel defining an inner surface and an outer surface, a first direction being a fluid flow direction along a length of the bare microfluidic channel and a second direction perpendicular to the first direction;
receiving operation parameters for a ciliated microfluidic channel formed from the bare microfluidic channel, the operation parameters including fluid viscosity and an opposing pressure gradient in an adverse direction to the fluid flow direction; and
determining cilia design parameters for the one or more cilia layers to be attached to and distributed over the inner surface, the cilia design parameters being determined from the incompressible Brinkman equation.

2. The method of claim 1 wherein motion of cilia in a traveling wave.

3. The method of claim 2 wherein motion of cilia in the one or more cilia layers is modeled as a longitudinal wave that travels in the first direction.

4. The method of claim 3 wherein motion of cilia in the one or more cilia layers is modeled such that the longitudinal wave remains in sync along the second direction.

5. The method of claim 2 wherein motion of cilia in the one or more cilia layers is modeled as a square wave, a sinusoidal wave, and/or a two-dimensional wave of any wave profile.

6. The method of claim 1 wherein the cilia design parameters include cilia density, cilia height, cilia width, and cilia spatial distribution.

7. The method of claim 1 wherein incompressible Brinkman equation is described by equation 1:
−μ∇²u(x,y,t)+∇p(x,y,t)+ζ(u(x,y,t)−ν(x,y,t))=−ΔP/L·eₓ∇·u(x,y,t)=0 (1)
wherein:
μ is the fluid viscosity;
u(x, y, t) is a fluid velocity field;
p(x,y, t) the pressure field; and
ζ is the Brinkman coefficient that relates permeability to fluid drag forces which can also be a function of position and time (i.e., ζ(x, y, t));
L is the length of the ciliated microfluidic channel being modeled;
ν(x, y, t) is a velocity field representing motion of cilia in the one or more cilia layers;
ΔP/L represents a uniform pressure gradient in the adverse direction to the fluid flow direction; and
eₓ is a unit vector in the flow direction.

8. The method of claim 7 wherein periodic boundary conditions are applied in the fluid flow direction and no-slip boundary conditions are applied in the second direction.

9. The method of claim 7 wherein the Brinkman coefficient is estimated from a total drag force inside the ciliated microfluidic channel.

10. The method of claim 1 wherein the microfluidic device is a cilia-driven inline microscale pumps.

11. The method of claim 1 wherein the microfluidic device is an inline microscale filter.

12. The method of claim 1 wherein the bare microfluidic channel has a cross-section that varies along the fluid flow direction.

13. A non-transitory computer-readable storage medium encoding instructions to execute the method of claim 1.

14. The microfluidic device made with the cilia design parameters determined by the method of claim 1.

15. The microfluidic device of claim 14 wherein the ciliated microfluidic channel has a cross-sectional area that is adjustable between a first cross-sectional area and a second cross-sectional area.

16. The microfluidic device of claim 14 wherein the ciliated microfluidic channel has a cross-section that varies along the fluid flow direction.

17. The microfluidic device of claim 14 wherein the bare microfluidic channel is composed of a chemically and biologically inactive material.

18. The microfluidic device of claim 17 wherein the chemically and biologically inactive material includes a component selected from the group consisting of acrylics, polysiloxanes, polycarbonates, linear low density polyethylene, acrylonitrile butadiene styrene, a cycloolefin copolymer, glass, mica, silica, semiconductor wafers, and combinations thereof.

19. The microfluidic device of claim 17 wherein the chemically and biologically inactive material includes a component selected from the group consisting of collagens, gelatin, hyaluronic acid, chitosan, heparin, alginate, fibrin, polyvinyl alcohol, polyethylene glycol, sodium polyacrylate, acrylate polymers, and copolymers thereof.

20. The microfluidic device of claim 14 wherein the bare microfluidic channel is composed of a hydrogel.

21. The microfluidic device of claim 14 wherein the cilia layers include artificial cilia composed of a magnetic material, and/or electrically or thermally driven artificial cilia composed of a smart material.

22. The microfluidic device of claim 14 wherein the a smart material is selected from the group consisting of liquid crystalline elastomers or metamaterials.

23. The microfluidic device of claim 14 wherein the cilia layers include tissue engineered biological cilia.

24. A system for designing a microfluidic device, the system comprising:
a computing device having a processor and memory in electrical communication with the processor, the computing device configured to execute steps of:
receiving an input design of a bare microfluidic channel to which a one or more cilia layers are to be added, the bare microfluidic channel having a predetermined cross section, the bare microfluidic channel defining an inner surface and an outer surface, a first direction being a fluid flow direction along a length of the bare microfluidic channel and a second direction perpendicular to the first direction;
receiving operation parameters for a ciliated microfluidic channel formed from the bare microfluidic channel, the operation parameters including fluid viscosity and an opposing pressure gradient in an adverse direction to the fluid flow direction; and
determining cilia design parameters for the one or more cilia layers to be attached to and distributed over the inner surface, the cilia design parameters being determined from the incompressible Brinkman equation.

25. The system of claim 24 wherein motion of cilia in a traveling wave.

26. The system of claim 25 wherein motion of cilia in the one or more cilia layers is modeled as a longitudinal wave that travels in the first direction.

27. The system of claim 26 wherein motion of cilia in the one or more cilia layers is modeled such that the longitudinal wave remains in sync along the second direction.

28. The system of claim 25 wherein motion of cilia in the one or more cilia layers is modeled as a square wave, a sinusoidal wave, and/or a two-dimensional wave.

29. The system of claim 24 wherein incompressible Brinkman equation is described by equation 1:
−μ∇²u(x,y,t)+∇p(x,y,t)+ζ(u(x,y,t)−ν(x,y,t))=−ΔP/L·eₓ∇·u(x,y,t)=0 (1)
wherein:
μ is the fluid viscosity;
u(x, y, t) is a fluid velocity field;
p(x,y, t) the pressure field; and
ζ is the Brinkman coefficient that relates permeability to fluid drag forces which can also be a function of position and time (i.e., ζ(x, y, t));
L is the length of the ciliated microfluidic channel being modeled;
ν(x, y, t) is a velocity field representing motion of cilia in the one or more cilia layers;
ΔP/L represents a uniform pressure gradient in the adverse direction to the fluid flow direction; and
eₓ is a unit vector in the flow direction.

30. The system of claim 29, wherein periodic boundary conditions are applied in the fluid flow direction and no-slip boundary conditions are applied in the second direction.
